(19) United States
(12) Patent Application Publication
(10) Pub. No.: US 2023/0132667 A1
(43) Pub. Date: May 4, 2023

(54) DRUG-LIKE MOLECULES AND METHODS FOR THE THERAPEUTIC TARGETING OF VIRAL RNA STRUCTURES

(71) Applicant: UNIVERSITY OF WASHINGTON, Seattle, WA (US)

(72) Inventors: Matthew Shortridge, Seattle, WA (US); Gabriele Varani, Seattle, WA (US); Venkata Vidadala, Seattle, WA (US)

(73) Assignee: UNIVERSITY OF WASHINGTON, Seattle, WA (US)

(21) Appl. No.: 17/999,545

(22) PCT Filed: May 25, 2021
(86) PCT No.: PCT/US2021/034076

§ 371 Date: Nov. 21, 2022

Related U.S. Application Data
(60) Provisional application No. 63/031,097, filed on May 28, 2020.

(51) Int. Cl. C07D 471/04; A61P 31/18

(52) U.S. Cl. CPC C07D 471/04; A61P 31/18

(57) ABSTRACT

Methods for identifying selective RNA-binding small molecules by NMR screening. The method provides a screening cascade to identify molecules that bind to an RNA structure, such as HIV TAR. Compounds that bind to structured RNAs and that are useful to disrupt the formation of RNA-protein complexes, such as P-TEFb-Tat-TAR complex.

### CROSS-REFERENCE TO RELATED APPLICATION

This application claims the benefit of U.S. Application No. 63/031,097, filed May 28, 2020, expressly incorporated herein by reference in its entirety.

### STATEMENT OF GOVERNMENT LICENSE RIGHTS

This invention was made with government support under Grant Nos. RO1 GM103834 and R35 GM126942, awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is UWOTL174223_Seq_List_FINAL_20210525_ST25.txt. The text file is 3 KB; was created on May 25, 2021; and is being submitted via EFS-Web with the filing of the specification.

### BACKGROUND OF THE INVENTION

Natural products and fully synthetic antibiotics are well-known class of compounds which target ribosomal RNA (rRNA) and elicit a therapeutic response. Examples include aminoglycosides and macrolides which inhibit protein translation and many more examples, including the oxazolidinone class of inhibitors of protein synthesis (e.g., linezolid). The successful targeting of rRNA suggests that targeting cellular RNAs, including messenger RNA (mRNA) and non-coding RNAs (ncRNAs) such as the ribosome, tRNA or newly discovered non coding RNAs, with small drug-like molecules could provide new therapeutic avenues to treating chronic and infectious diseases in humans, livestock and plants. While aminoglycosides, as well as other natural products, bind bacterial rRNA, these compounds have limited specificity and bind many RNAs irrespectively of sequence. Despite increased research efforts in both academic and biotech settings, the identification of potent, specific and pharmaceutically attractive small molecules which bind to RNA potently and specifically remains a very significant challenge.

Similar to proteins, most RNA sequences (e.g., tRNA, rRNA, riboswitches, ribozymes and many more) can fold into elaborate three-dimensional structures that provide binding sites for small molecule ligands; ribosomal RNA and, especially, riboswitches provide excellent examples of recognition of structured RNAs by small molecules. However, the elaborate three-dimensional and higher order folding observed in riboswitches, RNA enzymes and ribosomal RNAs, are not common in non-coding RNAs and mRNAs, which are less structured and coated in the cell with single strand RNA binding proteins (ssRBPs), such as hnRNPs and others, and typically have lower degree of structure in vivo than they do in experiments conducted in vitro. Simpler secondary structures are instead ubiquitous in non-coding RNAs and mRNAs, and well-known to perform regulatory functions, by providing binding sites for other RNAs, for RNA-binding proteins or by directly affecting access of the ribosome and of other translational initiation factors during initiation of protein synthesis or during RNA localization and stability, and other steps of RNA biogenesis, for example by regulating processing efficiency during mRNA splicing or 3′-end processing.

The RNA hairpin or stem-loop is the most common local secondary structure motif found in RNA sequences and can form within the context of much larger sequences (mRNAs) or as discreet, stand-alone functional structures (e.g., in microRNA precursor species). Many regulatory functions are associated with RNA stem-loops in the healthy and diseased state of cells. In microRNA precursors, for example, the stem-loop provides binding sites for the processing enzymes Drosha (with its co-factor in the microprocessor complex) and Dicer (with its co-factor TRBP) which generate the mature functional form of 20-22 nts. Within the 5′-UTRs of many mRNAs, for example in growth factors, housekeeping genes and many proto-oncogenes, formation of stable stem-loops, or hairpins, inhibits initiation of protein synthesis and reduces expression of the corresponding protein, particularly in proximity to the cap at the very 5′-end of a mRNA.

The basic RNA hairpin structure forms when a stretch of RNA nucleotides within the same RNA sequence contain two complementarity stretches of nucleotides with the potential to form Watson-Crick or wobble GU base pairs. When energetically favorable, these complementary regions fold onto themselves by forming hydrogen bonding and stacking interactions in an anti-parallel fashion to generate a double-stranded helical region (dsRNA, the ‘stem’). The complementary regions often leave unpaired nucleotides that form internal loops and bulges within an imperfect double helix. Formation of the double helix leaves unpaired nucleotides to form an apical loop where sequence complementarity does not exist. RNA stem-loop structures containing 3, 4 and 5 nt apical loops are common, with certain unique set of apical loop sequences (e.g., tetraloops) having high degree of structural stability. However, these single stranded loops can be as long as 15 nucleotides or more, as found in many microRNA precursor species. The generalized structure of an RNA hairpin is therefore a dsRNA helical region (the stem), with bulged or internal loop nucleotides interspersed within it, capped by an apical loop comprised of unpaired nucleotides, which can have varying length (see FIGS. 1A-1C).

The prevalence of the RNA hairpin structure amongst RNA sequences and their numerous functional roles suggest targeting such specific RNA structures could generate new leads for pharmaceutical development. Furthermore, these structures are likely to form even under conditions in vivo where more complex and less stable structures are less likely to form, because of their stability and local folding properties. Because they are associated with many biological functions in both healthy and diseased cellular states, stem-loops provide a large class of novel targets with biologically relevant function in diseases. However, specific targeting of these RNAs with drug-like molecules is believed to be very challenging, because they are so similar to each other and, it is believed, devoid of distinctive binding pockets. To date, most attempts at discovering small molecules that bind to RNA hairpins of bacterial, viral or mammalian origin have identified compounds with only weak affinity (μM) and/or highly charged basic molecules with little selectivity for their intended target sequence, or have pharmacological characteristics unlikely to lead to successful pharmaceutical applications. Therefore, it has been stated that the chances of targeting RNA hairpin structures selectively and potently with small molecules chemistry are low.

The transactivation response element of HIV (TAR) is a well-studied model system for understanding RNA-small molecule interactions. As shown in FIGS. 1A-1C, HIV TAR contains many structural features commonly found in RNA hairpins (dsRNA stem, bulge nucleotides, apical loop). The UCU bulge region of the TAR hairpin binds the arginine rich motif (ARM) of the HIV trans-activator protein Tat to facilitate recruitment of the super elongation complex (SEC) and enhance proviral transcription. Therefore, the Tat-TAR interaction is critical for viral replication and one of the most intensely studied protein-RNA complexes. Inhibiting the interaction between the TAT-ARM and the TAR bulge region has long been pursued to discover new anti-viral or latency reversal agents (Table 1).

TABLE 1
Examples of HIV TAR RNA binding small molecule compounds.

| Compound | Structure 5 |
|---|---|
| Neomycin | 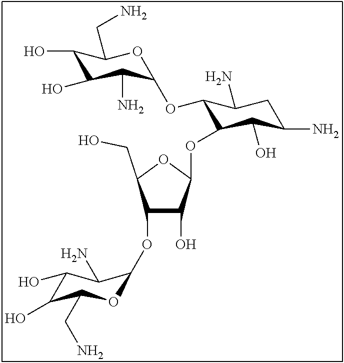 |
| RBT-550 | 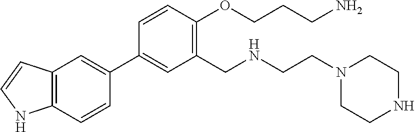 [SMILES: NCCCOc1ccc(-c2ccc3[nH]ccc3c2)cc1CNCCN1CCNCC1] |
| RBT-203 | 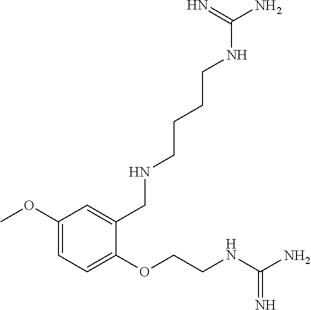 [SMILES: COc1ccc(OCCNC(=N)N)c(CNCCCCNC(=N)N)c1] |
| 104FA | 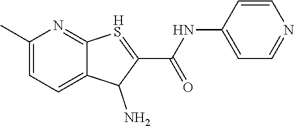 |
| Compound 3 | 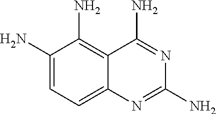 [SMILES: Nc1nc(N)c2c(N)c(N)ccc2n1] |
| Acetylpromazine | 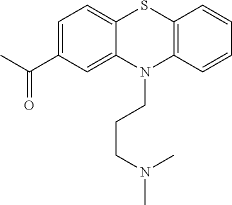 [SMILES: CC(=O)c1ccc2c(c1)N(CCCN(C)C)c1ccccc1S2] |

RNA hairpins (specifically HIV TAR) have been shown to be targetable with high affinity and specificity by using macrocyclic peptides. Arginine rich, macrocyclic peptides of 14 or 18 amino acids were synthesized to fold into stable anti-parallel beta-sheet hairpin structures, capped by a heterochiral D-Proline/L-Proline turn. These molecules penetrate eukaryotic cells and can target RNA hairpins inside cells, but lack the favorable pharmacologic properties associated with small drug-like molecules (delivery, localization, cell permeability, intracellular localization). Using this chemistry, structure-based approaches have generated ligands with low picomolar affinity and 10²-10⁶-fold binding selectivity relative to closely related RNA sequences and structures (FIG. 1C, Table 2).

TABLE 2
Macrocyclic Peptides that bind to HIV TAR RNA;
standard single letter amino acid identifiers are
used; lower case represents D-amino acids, dab is
diamino butyric acid, NOR is norarginine
(2-amino-4-guanidinobutanonic acid).

| | | |
|---|---|---|
| L50 | _{cyclo}(PRVRTRGKRRIRPp) | (SEQ ID NO: 1) |
| L22 | _{cyclo}(PRVRTR KGRRIRIp) | (SEQ ID NO: 2) |
| JB181 | _{cyclo}(P(dab)VRTRKGRRI(NOR)Ip) | (SEQ ID NO: 3) |

Although the pharmaceutical properties of these peptide macrocycles are unfavorable compared to traditional drug-like small molecules, these macrocycles can interrogate the biochemical and biological responses of putative RNA pharmaceutical targets. This is a non-trivial task as many RNA binding sites are dynamic until a binding ligand is identified, and free- and bound-forms of RNA can differ greatly from each other.

A good example of the change in RNA structure upon binding a protein or ligand is provided by the arginine rich motif of TAT binds the TAR bulge region and induces a large 3-dimensional structural change in the RNA hairpin relative to the free RNA structure (RMSD 4.7A) (FIG. 2). This structural rearrangement is recapitulated by the macrocyclic peptide JB181 (FIG. 2) and to a lesser extent by small molecules (FIG. 2) which also bind to the bulge region of TAR. The 3D structures reported in FIG. 2 were determined by nuclear magnetic resonance (NMR). In FIGS. 3A-3C, the NMR, ¹H-¹H 2D-TOCSY (Total COrrelation SpectroscopY) spectra are shown for each of the peptide and small molecule ligands reported in FIG. 2, when bound to HIV TAR. The NMR signal is sensitive to the chemical and magnetic environment of a molecule; therefore, ligands which induce similar changes in RNA structure show similar spectral ‘fingerprints’. The 3D structures of Tat- and JB181-bound HIV TAR are more similar to each other (RMSD 2.09 A) than to free RNA. The structural similarity between the two peptide bound structures is mirrored by the change in chemical shift induced by binding the ARM of TAT (FIG. 3A) and JB181 (FIG. 3B), relative to free HIV TAR (black peaks, FIGS. 3A-3C). In both cases, many of the same peaks experience changes in the same direction and similar magnitude. The small molecule RBT550 also induces large chemical shift changes relative to the free RNA but, unlike the peptide ligands, the small molecule does not induce the base triple involving U23/A27/U38, leading to a relatively large RMSD difference between the small molecule bound structure and the peptide bound structures (RMSD 3.07A). This TOCSY fingerprinting approach is therefore valuable when discovering new ligand bound structures to RNA.

Despite the unprecedented binding affinity and specificity of the new peptide ligand (JB181), the macrocycle only moderately inhibits Tat-dependent reactivation in cells and recruitment of P-TEFb to TAR (150). While determining the structure of JB181 bound to HIV TAR, the crystal structure of TAR bound to components of the Super Elongation Complex (SEC) was reported. Aligning the NMR structure of the JB181-TAR complex reveled the ligand induces a structure in the TAR loop that closely mimics the P-TEFb/Tat1:57/AFF4/TAR complex (FIGS. 4A-4C). This was not entirely surprising since, as shown in FIGS. 2 and 3A-3C, JB181 induced a structure in TAR similar to what is induced by the wild-type ARM of the TAT peptide. The JB181 ligand does not inhibit binding to this PTEFb/AFF4/TAT complex; rather, the PTEFb/AFF4/TAT complex still binds to the HIV TAR:JB181 complex. A working hypothesis emerging from these results is that the ARM of TAT is important, but not essential for the formation of the full complex of TAR/PTEFb/AFF4/TAT. Rather, the TAT ARM induces a specific structure in the TAR hairpin which allows the complex to the bind the TAR loop with high affinity. From these results, it was suggested that JB181 induces the same structure in the HIV TAR RNA such that the TAR loop residues are still able to bind to the PTEFb/AFF4/TAT complex with high affinity, while displacing the TAT-ARM (FIGS. 5A and 5B), perhaps explaining why many small molecules which bind to the bulge of HIV TAR with high affinity do not show significant TAT dependent antiviral activity.

As discussed above and detailed in many review articles, the HIV TAR hairpin has been a long-standing target and model system for RNA-small molecule discovery efforts for antiviral development. Though many compounds for HIV TAR have been reported, no other compound has the degree of binding selectivity reported with the JB181 peptide. Furthermore, the majority of small molecules reported to bind to HIV TAR target the U23/C24/U25 trinucleotide bulge; however, targeting the bulge may induce a structure within the RNA hairpin that is still recognized by the SEC through binding the loop residues of TAR. Palbocicilib is one of three compounds (FIG. 6) currently on the market that target CDK4/CDK6 enzymes in HR-Positive HER2-negative breast cancer. Recent reports suggested the compounds targeting CDK6 enzymes have antiviral activity against both HIV and Zika viruses, although the mechanism of action was not established. These compounds were active against HIV in a spreading assay, with IC₅₀ values below 250 nM 7 days post infection after single dose administration (FIG. 7).

Despite the advances in the identification of small molecules targeting structured RNAs, a need exists for improved methods for identification of small molecules that target structured RNAs and for the identification and optimization of specific small drug-like molecules targeting structured RNAs. More specifically, a need exists for high affinity small molecules that bind structured RNAs, such as HIV TAR, and disrupt the formation of RNA-protein complexes, such as the P-TEFb-TAT-TAR. The present invention seeks to fulfill this need and provides further related advantages.

### SUMMARY OF THE INVENTION

In one aspect, the invention provides compounds that bind to structured RNAs and that are useful to disrupt the formation of RNA-protein complexes.

In one embodiment, the compound has formula (I):

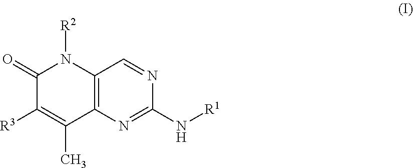

or a pharmaceutically acceptable salt thereof, wherein R¹ is selected from the group consisting of

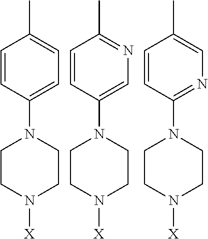

wherein X is hydrogen, C1-C6 alkyl, or C(═O)C1-C6 alkyl;

R² is selected from the group consisting of hydrogen, C1-C6 alkyl, and C3-C6 cycloalkyl; and

R³ is selected from the group consisting of hydrogen and or C(═O)C1-C6 alkyl.

In certain embodiments, X is selected from the group consisting of hydrogen, methyl, ethyl, n-propyl, i-propyl, and C(═O)CH₃.

In certain embodiments, R² is selected from the group consisting of hydrogen, methyl, ethyl, n-propyl, i-propyl, cyclopropyl, cyclobutyl, cyclopentyl, cyclohexyl, and C(═O)CH₃.

In certain embodiments, R³ is selected from the group consisting of hydrogen and C(═O)CH₃.

Representative compounds include the compounds of Table 4, or pharmaceutically acceptable salts thereof.

In another aspect, the invention provides pharmaceutical compositions. In one embodiment, the pharmaceutical composition comprises a compound of the invention as described herein, or a pharmaceutically acceptable salt thereof, and a pharmaceutically acceptable carrier.

In further aspects, methods for using the compounds of the invention are provided.

In one embodiment, the invention provides a method for inhibiting the binding of human positive transcription elongation factor complex (P-TEFb) to HIV-1 trans-activation response element (HIV TAR) in a subject, comprising administering to a subject in need thereof an effective amount of a compound as described herein, or a pharmaceutically acceptable salt thereof.

In another embodiment, the invention provides a method for disrupting formation of the P-TEFb-Tat-TAR complex in a subject, comprising administering to a subject in need thereof an effective amount of a compound as described herein, or a pharmaceutically acceptable salt thereof.

In a further embodiment, the invention provides a method for inhibiting miRNA processing in a subject, comprising administering to a subject in need thereof an effective amount of a compound as described herein, or a pharmaceutically acceptable salt thereof.

In yet another embodiment, the invention provides a method for treating a disease, disorder, or condition treatable by inhibiting miRNA processing, comprising administering to a subject in need thereof a therapeutically effective amount of a compound as described herein, or a pharmaceutically acceptable salt thereof.

In another embodiment, the invention provides a method for treating a disease, disorder, or condition treatable by inhibiting mRNA function, including but not limited to translation, alternative splicing, stability, comprising administering to a subject in need thereof a therapeutically effective amount of a compound as described herein, or a pharmaceutically acceptable salt thereof.

In a further embodiment, the invention provides a method for treating a disease, disorder, or condition treatable by inhibiting the function of a noncoding RNA gene, comprising administering to a subject in need thereof a therapeutically effective amount of a compound as described herein, or a pharmaceutically acceptable salt thereof.

In other aspects, the invention provides NMR methods useful for identifying targetable and druggable RNA structures.

In one embodiment, the invention provides a method of identifying targetable and druggable RNA secondary structures in a viral RNA, viral RNA, non-coding RNA or mRNA, comprising:

contacting a primary miRNA sequence, a precursor miRNA sequence, a mRNA, viral RNA, or a noncoding RNA sequence with a ligand and determining by NMR spectroscopy whether the ligand binds to the RNA sequence.

In another embodiment, the invention provides a method of identifying a mRNA, miRNA or non-coding RNA ligand, comprising:

contacting an RNA sequence, comprising an RNA secondary structure, with a candidate ligand; and

determining by NMR spectroscopy whether the ligand binds to the RNA sequence, wherein binding indicates a ligand which binds to the RNA.

In a further embodiment, the invention provides a method of identifying a miRNA ligand, comprising:

contacting a primary miRNA sequence or a precursor miRNA sequence with a candidate ligand; and

determining by NMR spectroscopy whether the ligand induces a conformation change in the primary miRNA sequence or precursor miRNA sequence, wherein the conformation change indicates the ligand binds to the primary miRNA sequence or the precursor miRNA sequence.

In certain embodiments of the NMR methods of the invention, the ligand is a compound of the invention as described herein, or a pharmaceutically acceptable salt thereof.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A-1C illustrate common features of RNA stem-loop (hairpin) structures. FIG. 1A illustrates secondary structure of the HIV1-TAR RNA which shows common secondary structure elements found in RNA; including two double stranded stem regions, a three-nucleotide bulge and a six-nucleotide apical loop capping the structure. FIGS. 1B and 1C illustrate the three-dimensional structure of the HIV TAR RNA in the absence of any ligand (PDB 1ARN) and in the presence of the macrocyclic peptide ligand JB181 (PDB 6D2U), respectively. While the secondary structure remains the same regardless of whether a ligand is bound or not, the three-dimensional structure changes with different ligands. FIG. 3C shows how JB181 makes specific contacts to each stem region, the bulge and apical loop (PDB 6D2U). The binding site of a ligand is not necessarily localized to a single site on a secondary structure element, but rather encompasses regions that folds around the ligand, as observed in more extensive ways in riboswitches. This observation supports the need for accurate structural investigation. Relying on modeling is not possible because blind predictions of small molecule binding sites in RNA fail to provide high resolution accuracy, even in relatively favorable cases like riboswitches and aptamers.

FIG. 2 compares free and ligand-bound 3D structures of HIV TAR determined by NMR: the free HIV TAR RNA (PDB:1ANR); bound to the TAT-ARM (PDB:6MCE); to JB181 (PDB:6D2U); and to the small molecule RBT550 (PDB:IUTS). The U23/C24/U25 bulge is the main binding site for both peptides and small molecule ligands (as listed in Tables 1 and 2). The two peptide bound structures are more similar to each other and more divergent from the free structure, compared to the RBT550 structure (see table inlay for pairwise RMSD values). These differences in structure are related to induced changes in the UCU bulge and the formation (or not) of the U23/A27/U38 base triple (found only in the two peptide-bound structures).

FIGS. 3A-3C compare of TOCSY spectra for HIV TAR bound to different ligands. Similar to the heteronuclear ¹H-¹⁵N HSQC for proteins, the 2D ¹H-¹H TOCSY spectra show through bond correlations between the pyrimidine H5 and H6 protons of unique bases and provide a ‘fingerprint’ on how different compounds bind RNA. FIG. 3A shows the overlay of the free HIV-TAR (black) TOCSY and the Tat-ARM bound (grey) spectrum. FIG. 3B shows the spectrum of JB181-bound TAR (grey). FIG. 3C shows TAR bound to RBT-550 (grey). The large chemical shift changes in the bound spectra compared to the free spectrum for each of the ligands is indicative of strong interactions in all cases. However, the pattern of chemical shift changes also shows how similarly the ligands bind to the RNA structure mimicking the RMSD values in the 3D structure from FIG. 2. Each HIV TAR sample was prepared to 0.50 mM in 50 mM potassium phosphate buffer with 50 mM sodium chloride added. Ligands were titrated to 2× foldexcess (1 mM) over the RNA, and data were collected at 25° C. at 800 MHz.

FIG. 4A illustrates that the HIV TAR loop residues (nts 26-39), as observed in the TAR:PTEFb structure (PDB: 6CYT), were aligned to the same residues in the TAR:JB181 structure (PDB: 6D2U) using the Pymol align feature, resulting in an RMSD of 1.49A. The TAR:JB-181 structure contains a 29 nt hairpin and includes the UCU bulge, while the TAR:P-TEFb complex contains a shortened bulge-less HIV TAR, along with the proteins Tat, AFF4, Cyt1 and CDK9. FIGS. 4B and 4C are close-up views of potential contacts with the P-TEFb complex and JB181-bound TAR. These models help explain why the peptide-bound RNA can bind to the P-TEFb complex and suggest the high affinity peptide induces similar structures in HIV TAR as P-TEFb, while displacing the arginine rich motif of TAT.

FIGS. 5A and 5B illustrates the TAR complex formed in the presence of the JB-181 peptide because interactions between the TAR-loop and CycT1 are retained. FIG. 5A is a close up view of HIV TAR bound to the P-TEFb complex (PDB 6CYT); highlighted in the circle are the CycT1 residues which bind to the TAR loop through a basic patch on the CycT1 surface (dark grey). FIG. 5B shows the aligned P-TEFb-RNA complex from FIG. 4A represented here with the PTEFb protein in surface representation; the JB181-TAR complex structure is shown as cartoon and sticks. A small molecule which bind to the apical loop of HIV TAR may disrupt the HIV TAR-P-TEFb interaction, while the JB181 peptide or other ligands which bind to the bulge region is unlikely to do so.

FIG. 6 illustrates the chemical structures of the three FDA-approved CDK4/CDK6 ligands.

FIG. 7 compares antiviral activity of CDK4/CDK6 inhibitors: spreading infection assays in CD4+ T-cells, which were infected with GFP+ HIV, then drugs were added 24 hours later (each data point is a dose from 2-fold dilution starting from 500 μM to 0.25 μM, right to left). Thus, infection was allowed to initiate but spread would be prevented if the drugs stopped the replication cycle. Data represent the % of control at 3 different time points post infection. Compound MSGV-100 is Abemaciclib, MSGV-200 is Ribociclib, and MSGV-300 is Palbociclib

FIG. 8 illustrates the single point ligand-detect primary NMR screening step (stage 1). Identification of Palbociclib as a molecule that binds to HIV TAR: top, reference spectrum of 100 μM free ligand; middle, spectrum of Palbociclib upon addition of pre-miR-21 to the free ligand; and bottom, spectrum of Palbociclib upon addition of HIV TAR to the free ligand. The ligand signals are identified by arrows and other buffer components are labeled in the spectra. This experiment provides a rapid and robust method to detect RNA and protein binding compounds. The decrease in ligand signal is due to the increase in rotational correlation time (Tc) of the ligand when bound to the RNA target and is revealed by relaxation-editing of the spectra through the NMR pulse sequence. Compounds that do not show binding (non-binding control, DSA) do not show a decrease in the NMR signal.

FIGS. 9A-9D illustrate the selectivity screening for RNA binding (stage 2). FIG. 9A shows the structure of Palbociclib. FIG. 9B shows spectra normalized to the non-binding internal reference standard sodium 4,4-dimethyl-4-silapentane-1-sulfonate (DSA; 9 protons) and intensities were plotted as a function of RNA concentration, to generate a binding isotherm from which approximate binding constants can be generated by curve fitting (0 HIV TAR, ⋅Pre-miR-21). FIG. 9C shows 100 μM Palbociclib titrated with HIV TAR RNA (0-504). FIG. 9D shows 100 μM Palbociclib titrated with pre-miR-21 RNA (0-5 μM). All spectra were collected at 800 MHz under ‘high salt’ conditions (50 mM d9-deuterated bis-Tris buffer at pH 6.5, containing 200 mM NaCl, 50 mM KCl and 4 mM MgCl₂ and 37° C.).

FIGS. 10A and 10B illustrate the target-detected screening methods (stage 3). Binding of Palbociclib to different RNAs occurs with different binding characteristics, which are reflected in the NMR spectra of the RNA. All TOCSY spectra were collected at 800 MHz under high salt conditions at 37° C. 250 μM RNA (black) was titrated with Palbociclib (grey) until saturation was reached (as established from the absence of further changes in the spectra) and changes in chemical shifts were recorded. FIG. 10A illustrates that HIV TAR shows dramatic chemical shift changes and ‘slow exchange’ behavior between conformations, with regular peak shape for all signals, indicative of high affinity and site-specific binding, whereas pre-miR-21. FIG. 10B shows much smaller chemical shift changes and irregular peak shapes, indicative of much weaker affinity and a poorly defined interaction site reflective of non-specific binding. These data mirror the 1D-¹H binding data of FIG. 9 and demonstrate that Palbociclib binds specifically to HIV TAR compared to other hairpin RNA structures, like pre-miR-21.

FIGS. 11A and 11B illustrate the target-detected screening methods (stage 3). Binding of Palbociclib to HIV TAR under different pH conditions: 50 mM sodium acetate at pH 4.5 (FIG. 11A) and 50 mM bis-Tris at pH 6.5 (FIG. 11B). All TOCSY spectra were collected at 800 MHz under high salt conditions at 37° C. 250 μM RNA (black) was titrated with Palbociclib (grey) until saturation was reached (as established from the absence of further changes in the spectra) and changes in chemical shifts were recorded.

FIGS. 12A-12C compare orthogonal affinity measurement (stage 4). Comparison of 2-AminoPurine-labeled HIV TAR binding assays (top) with target-based NMR assays monitoring imino chemical shifts (bottom) for Palbociclib, Ribociclib, and Abemaciclib. With Palbociclib, the 2AP assay show a rapid increase in fluorescence signal indicating an increase in base stacking upon binding, followed by a decrease in signal at higher concentrations of ligand. This biphasic binding curve suggests the presence of two binding sites, a high affinity site with apparent K_{D} of 104±80.2 nM and a lower affinity site with apparent K_{D}>700 nM. The imino resonance signals for HIV TAR upon binding to Palbociclib, Ribociclib, and Abemaciclib, which only display the second weaker binding mode are consisted with the binding affinity estimated from the fluorescence assay (spectra are broadened by Ribociclib and Abemaciclib, while slow exchange behavior is observed for Palbociclib).

FIGS. 13A and 13B show NMR-based identification of features required for binding to RNA: many intermolecular NOE interactions are observed between HIV TAR and Palbociclib, many involving the N8 cyclopentane ring (Hv,w,x,y) and methyl protons (Hg, Hi), identifying potential drivers of the strong interaction with RNA. The boxed intermolecular NOEs reveal the many contacts observed between the cyclopentane ring and the sugar region of the RNA spectrum. Each spectrum was collected at 800 MHz with 300 ms mixing time; the sample contained 500 μM RNA and 750 μM Palbociclib in 50 mM d9-bisTris pH 6.5, 50 mM NaCl in 99.99% D₂O.

FIGS. 14A and 14B compares the unique structure fingerprint of Palbociclib: similar to FIGS. 3A-3C, the TOCSY spectrum provides a structural fingerprint of how ligands bind to RNA. Similar changes in TOCSY chemical shift values suggest ligands adopt similar structures. For Palbociclib (FIG. 14B), the large chemical shift changes between free (black) and bound (grey) spectra, compared (FIG. 14A) with the large chemical shift changes between the JB181 bound spectrum (grey) suggest Palbociclib induces a new, so far uncharacterized structure of HIV TAR.

FIGS. 15A and 15B compare models of Palbociclib bound to HIV TAR. The ligand makes significant contacts with the upper loop region, consistent with the data of FIGS. 13A and 13B, suggesting the compound binds to the apical loop rather than the UCU bulge as most other TAR ligands characterized so far: surface rendering (FIG. 15A) and low energy (FIG. 15B) models.

FIGS. 16A-16C illustrate the disruption of the PTEFb-TAR complex by Palbociclib: based on the model of FIGS. 15A and 15B, the small molecule appeared to bind to a pocket at or near the site of interaction between P-TEFb and HIV TAR. Consistent with this hypothesis, the small molecule reduces the affinity between HIV TAR and the core Super Elongation Complex (P-TEFb/AFF4/Tat): 0.50 nM of 5′ end ³²P labeled HIV TAR was incubated with 0-90 nM of the preformed P-TEFb/AFF4/Tat complex (by serial dilution) (FIG. 16A); Palbociclib (10.0 nM) was pre-incubated with the HIV TAR RNA prior to binding (0-90 nM of the P-TEFb/AFF4/Tat complex (by serial dilution) (FIG. 16B); and both assays were repeated in duplicate and resolved on 6% native acrylamide gels; bands were quantified with ImageJ and plotted vs complex concentration (FIG. 16C). The binding affinity (K_{D}) was measured to be 0.34±0.09 nM for the free-HIV TAR RNA (FIG. 16A), but it was reduced to 35.6±10 nM when 10.0 nM Palbociclib was preincubated with the free RNA prior to complex formation (FIG. 16B).

FIGS. 17A and 17B compare the structure of Palbociclib and a model for compound 4 bound to the catalytic site of Cdk6. FIG. 17A shows that the cyclopentane ring of Palbociclib mimics the ribose of the adenosine triphosphate substrate and binds into the hydrophobic pocket created by the flexible loop region of the kinase (dashed arrow) (PDB 5L2I). FIG. 17B shows the predicted pose for compound 4 modeled into the Palbociclib binding site. In docking studies, the bridge NH between the pyrimidine and pyridine moieties attempts to maintain the hydrogen bond with the backbone of Val101, but the pyrido[3,2,D]pyrimidin-6-one core structure in compound 4 flips the cyclopentane ring to occupy the pocket typically filled with the acetate group on Palbociclib (solid arrow). This ‘flip’ reduces predicted affinity for CDK6 enzyme to the mM range for Compound 4 yet Compound 4 retains all the key structural elements important for RNA binding. The acetate group on Compound 4 was removed to improve synthetic accessibility.

FIG. 18 illustrates the structure of the designed core molecules. The different sub-structures shown in FIG. 20 can be attached at R₁. Formula 1 is a substituted pyrido[3,2,D]pyrimidin-6-one core structure.

FIG. 19 illustrates sub-structures used in the generation of the RNA binding series.

FIG. 20 summarizes an example of NMR ligand detected binding curves for compounds with formula 1 structure (class 1). The pyrido[3,2,D]pyrimidin-6-one core structure improved binding affinity for both pre-miR21 (open symbols) and HIV TAR (closed symbols). NMR binding affinities for Palbociclib (squares) bound to HIV TAR (⋅) were fit to an apparent K_{D} of 0.3 μM and, for pre-miR-21 (∘) with a K_{D} of >4.4 μM. For compound 4 (circles), affinities improved to 0.1 μM for HIV TAR (⋅) and to 0.4 μM for pre-miR-21 (∘). The Bₘₐₓ values also increased for both RNAs, indicating reduced non-specific binding to RNA (compare squares and circles).

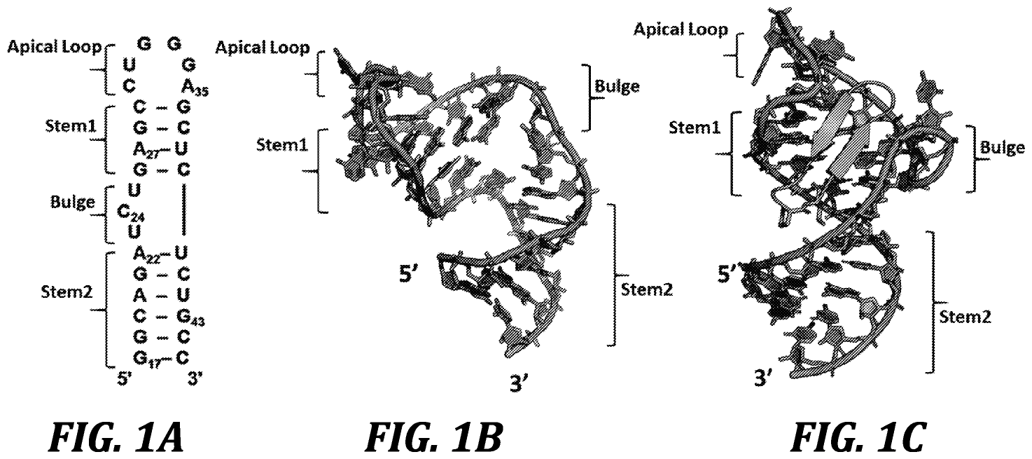

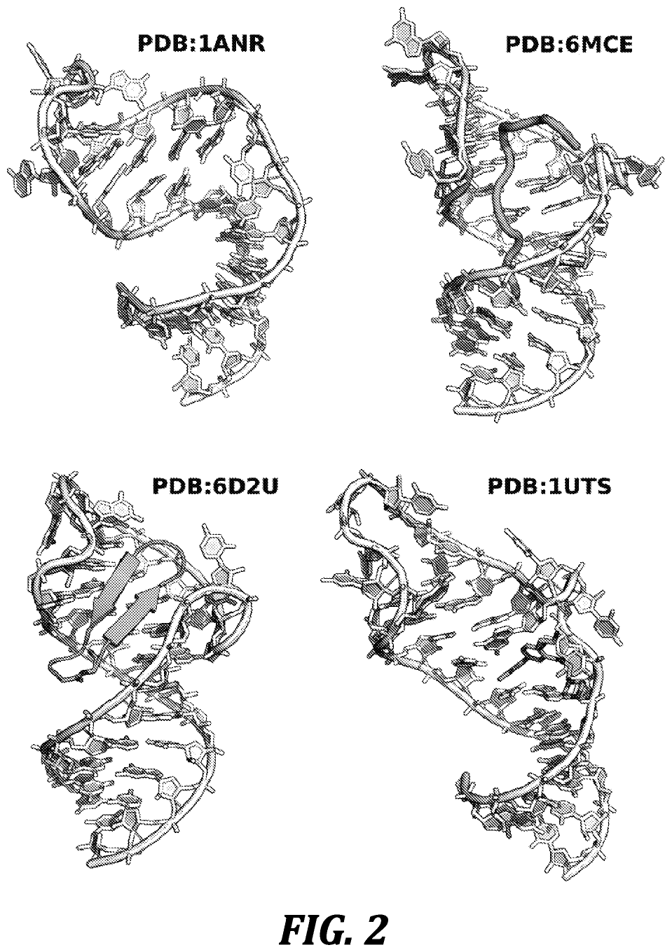

### DETAILED DESCRIPTION OF THE INVENTION

In one aspect, the invention provides a method for identifying selective RNA-binding small molecules by NMR screening. The method provides a screening cascade to identify molecules that bind to an RNA structure, such as HIV TAR.

In another aspect, the invention provides compounds that bind to structured RNAs and that are useful to disrupt the formation of RNA-protein complexes.

RNA-Binding Compounds

In one aspect, the invention provides compounds that bind to structured RNAs and that are useful to disrupt the formation of RNA-protein complexes.

In one embodiment, the compound has formula (I):

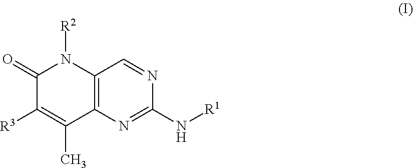

or a pharmaceutically acceptable salt thereof, wherein R¹ is selected from the group consisting of

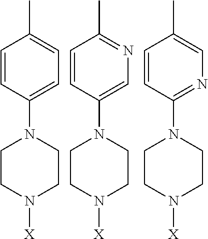

wherein X is hydrogen, C1-C6 alkyl, or C(═O)C1-C6 alkyl;

R² is selected from the group consisting of hydrogen, C1-C6 alkyl, and C3-C6 cycloalkyl; and

R³ is selected from the group consisting of hydrogen and or C(═O)C1-C6 alkyl.

In certain embodiments, R¹ is

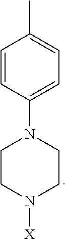

In other embodiments, R¹ is

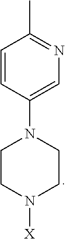

In certain embodiments, X is selected from the group consisting of hydrogen, methyl, ethyl, n-propyl, i-propyl, and C(═O)CH₃.

In certain embodiments, R² is selected from the group consisting of hydrogen, methyl, ethyl, n-propyl, i-propyl, cyclopropyl, cyclobutyl, cyclopentyl, cyclohexyl, and C(═O)CH₃.

In certain embodiments, R³ is selected from the group consisting of hydrogen and C(═O)CH₃.

Representative compounds include the compounds of Table 4, or pharmaceutically acceptable salts thereof.

In another aspect, the invention provides pharmaceutical compositions. In one embodiment, the pharmaceutical composition comprises a compound of the invention as described herein, or a pharmaceutically acceptable salt thereof, and a pharmaceutically acceptable carrier.

Methods for Using RNA-Binding Compounds

In further aspects, methods for using the compounds of the invention are provided.

In one embodiment, the invention provides a method for inhibiting the binding of human positive transcription elongation factor complex (P-TEFb) to HIV-1 trans-activation response element (HIV TAR) in a subject, comprising administering to a subject in need thereof an effective amount of a compound as described herein, or a pharmaceutically acceptable salt thereof.

In another embodiment, the invention provides a method for disrupting formation of the P-TEFb-Tat-TAR complex in a subject, comprising administering to a subject in need thereof an effective amount of a compound as described herein, or a pharmaceutically acceptable salt thereof.

In a further embodiment, the invention provides a method for inhibiting miRNA processing in a subject, comprising administering to a subject in need thereof an effective amount of a compound as described herein, or a pharmaceutically acceptable salt thereof.

In yet another embodiment, the invention provides a method for treating a disease, disorder, or condition treatable by inhibiting miRNA processing, comprising administering to a subject in need thereof a therapeutically effective amount of a compound as described herein, or a pharmaceutically acceptable salt thereof.

In another embodiment, the invention provides a method for treating a disease, disorder, or condition treatable by inhibiting mRNA function, including but not limited to translation, alternative splicing, stability, comprising administering to a subject in need thereof a therapeutically effective amount of a compound as described herein, or a pharmaceutically acceptable salt thereof.

In a further embodiment, the invention provides a method for treating a disease, disorder, or condition treatable by inhibiting the function of a noncoding RNA gene, comprising administering to a subject in need thereof a therapeutically effective amount of a compound as described herein, or a pharmaceutically acceptable salt thereof.

In other aspects, the invention provides NMR methods.

In one embodiment, the invention provides a method of identifying targetable and druggable RNA secondary structures in a viral RNA, viral RNA, non-coding RNA or mRNA, comprising:

contacting a primary miRNA sequence, a precursor miRNA sequence, a mRNA, viral RNA, or a noncoding RNA sequence with a ligand and determining by NMR spectroscopy whether the ligand binds to the RNA sequence.

In another embodiment, the invention provides a method of identifying a mRNA, miRNA or non-coding RNA ligand, comprising:

contacting an RNA sequence, comprising an RNA secondary structure, with a candidate ligand; and

determining by NMR spectroscopy whether the ligand binds to the RNA sequence, wherein binding indicates a ligand which binds to the RNA.

In a further embodiment, the invention provides a method of identifying a miRNA ligand, comprising:

contacting a primary miRNA sequence or a precursor miRNA sequence with a candidate ligand; and

determining by NMR spectroscopy whether the ligand induces a conformation change in the primary miRNA sequence or precursor miRNA sequence, wherein the conformation change indicates the ligand binds to the primary miRNA sequence or the precursor miRNA sequence.

In certain embodiments of the NMR methods of the invention, the ligand is a compound of the invention as described herein, or a pharmaceutically acceptable salt thereof.

As used herein, the term “NMR spectroscopy” refers to nuclear magnetic resonance spectroscopy experiments including, but not limited to, 1D ¹H and 2D ¹H-¹H TOCSY, ¹H-¹H NOESY, ¹H-¹H EXSY, ¹⁵N-¹H HSQC, and ¹³C-¹H HSQC experiments.

As described herein, Palbociclib is demonstrated as a high affinity structure for binding to certain RNA hairpins. Palbociclib binds to HIV TAR RNA with an apparent K_{D} of 104±80.2 nM, specifically recognizes the apical loop, induces a new structure in TAR and disrupts the formation of the PTEFb-TAR complex. The affinity of the TAR-Palbociclib complex is comparable to the reported CDK6-Palbociclib affinity (K_{D} 60 nM). The structure-based design methods described herein were used to optimize affinity towards HIV TAR while simultaneously reduce affinity towards the CDK enzymes. This information, coupled with changes in the dynamic signature of the RNA that results from the binding event, was used to identify and develop small drug-like molecules that specifically target RNA stem-loops and other structured RNAs. As described herein, the method was used to identify a set of compounds selective for HIV TAR.

Methods for Identifying Selective RNA-binding Small Molecules by NMR Screening

In one aspect, the invention provides a method that integrates RNA structure and dynamic information with NMR-based screening and structure-based optimization to overcome these challenges. This approach allows for identifying and optimizing new compounds robustly, leading to small molecules that target RNA selectively. The approach can be divided into 4 main steps (Target Selection, Lead Discovery, Lead Optimization and Activity Testing). The Target Selection step is crucial: in-depth structure and dynamic analysis of RNA target by probing target with RNA macrocycles prior to small molecule discovery greatly enhances the chance of success in identifying active compounds. Lead discovery: many screening methods can discover compounds binding to a biomolecular target; as described herein NMR is used as it provides direct structural information in addition to binding (small molecule ligand screening cascade). Lead optimization: a structure-based approach to optimize ligands that bind to RNA. Using NMR, we derive ligand bound co-structures to help guide the synthesis of new derivative molecules. Activity testing: biochemical assays are critical to identifying compounds with cellular activity.

This above approach can be applied to any RNA sequence starting from any chemical library and can be used to prioritize ligands within any chemical library for structure- or SAR-based hit and lead optimization. The approach is broken down into five distinct stages which, when followed, identify compounds that bind to structured RNAs and can be used for follow-up rounds of structure-based optimization. An example of a structure-based optimization approach is also described below. Components of both the small molecule discovery and structure-based optimization methods can be used for stand-alone analysis of small molecule RNA interactions.

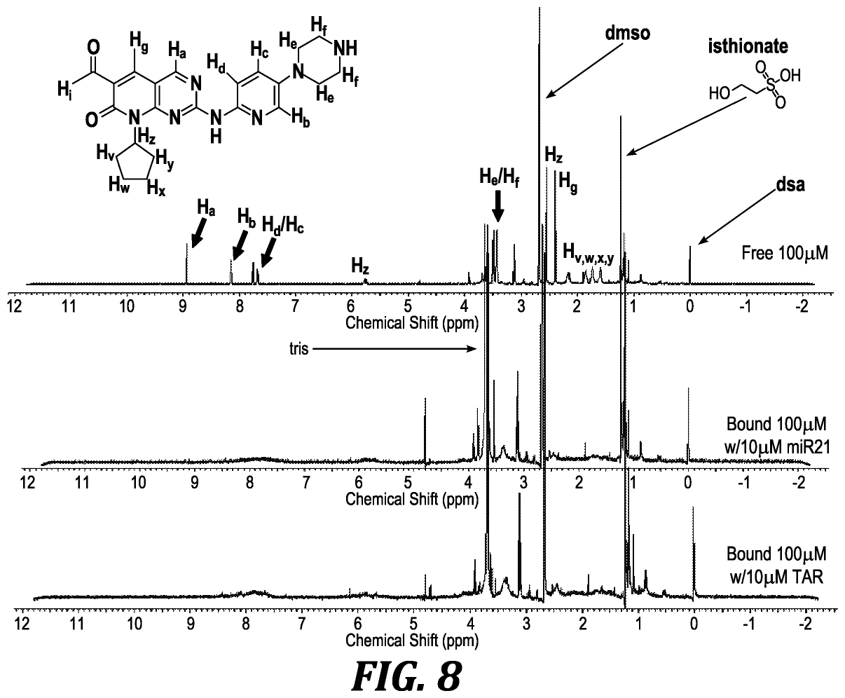

For initial screening and hit identification, a relaxation edited ligand-detect method is used to detect binding under low salt conditions. These initial conditions are chosen to facilitate the discovery of even low affinity compounds with favorable chemical characteristics. Binding compounds are identified through changes in peak height when comparing spectra for RNA-free and RNA-bound ligand (FIG. 8). The change in peak height is proportional to the increase in ligand line width, compared to the free small molecule, which occurs upon binding to the larger biomolecule. This NMR method is agnostic to biomolecular target, as it only depends on the longer rotational correlation time (T_{c}) of the small molecule bound in a complex with a much larger biomolecular target, which has a much shorter correlation time; other NMR methods (e.g., STD) can be used as well.

The method for identifying RNA binding compounds uses this screening method as an initial filter, because it provides a rapid approach to screening relatively large libraries of RNA binding compounds.

Stage 1. The first step in the screening cascade is a single point measurement of binding. The free ligand linewidths of compounds dissolved at concentration of 100 μM were quantified and compared to the ligand linewidths observed after addition of 10 μM RNA target. The concentrations for both ligand and target molecules were selected to maximize the likelihood of detecting even weak binding compounds, while minimizing background signal from the RNA which could potentially overlap with the small molecule signal.

Illustrative examples of this single point measurement are provided for three functionally related compounds, the FDA-approved breast cancer drugs Palbociclib, Ribociclib, and Abemaciclib, which were examined for binding to HIV TAR RNA, a well-known target of many anti-viral screening campaigns. Line-broadening experiments are dependent on molecular weight; therefore, to directly compare affinity, the use of target molecules with comparable molecular weight and shape is required.

In the initial NMR relaxation assay, a single point binding assay, two RNAs studied (TAR and pre-miR-21, which were studied as a control) showed a clear response in binding Palbociclib, as indicated by the obvious decrease in free ligand signal (FIG. 8) under the conditions of the experiment (100 μM small molecule in low salt buffer, 10 μM RNA). The buffer components remain sharp and well-resolved, showing the method can quickly triage binding responses using a binary yes/no binning system. Under these conditions, the other two compounds, Ribociclib and Abemaciclib, showed binding responses to both RNAs as well.

Importantly, these initial assays are done in low salt screening buffer to maximize the likelihood of identifying any hit, regardless of affinity. However, the low salt conditions do not allow evaluation of RNA selectivity, because they are permissive of electrostatic-driven binding (i.e., interactions driven primarily by charge involving basic small molecules binding to the strongly negatively charged nucleic acids regardless of sequence or structure). Many RNA screening campaigns have reported the identification of RNA binding molecules, for example natural products and ribosomal ligands, while cellular screen have discovered molecules with unspecified cellular targets.

The rapid comparison between free and bound spectra of a small molecule, requiring <6 mins per molecule (potentially less than 1 min/molecule if compound mixtures are used), clearly distinguishes binding from non-binding ligands.

Stage 2. The second step in the screening cascade involves measurements under high salt conditions, closer to the cellular milieu, on individual compounds to establish that binding is not driven by electrostatic interactions. Many RNA-binding molecules described in the literature have reduced binding to RNA under high salt conditions, as prevalent in the cell, because the interactions are driven by electrostatics; the negative charge of the RNA that makes it prone to non-specific interactions with basic ligands.

The high salt screening step allows the identification of more attractive RNA binding compounds from less attractive ligands whose affinity for RNA is driven by electrostatics but can also be used for quantification of binding affinities. In the example that follows, we show that Palbociclib binds to HIV TAR with much greater affinity than pre-miR-21, in fact >100-fold stronger, it essentially only binds TAR.

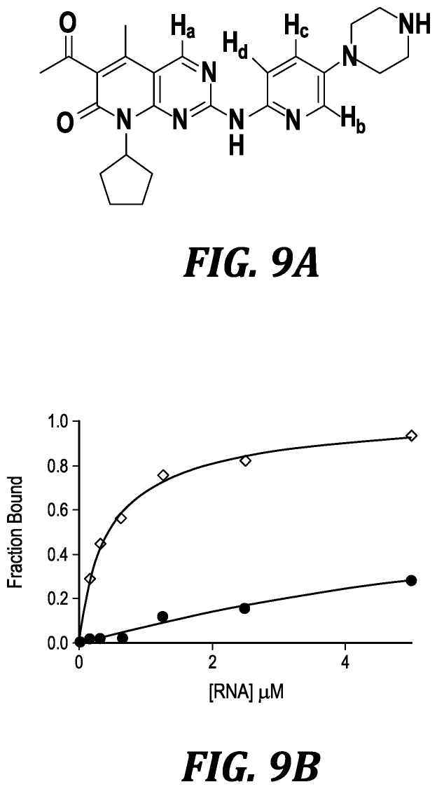

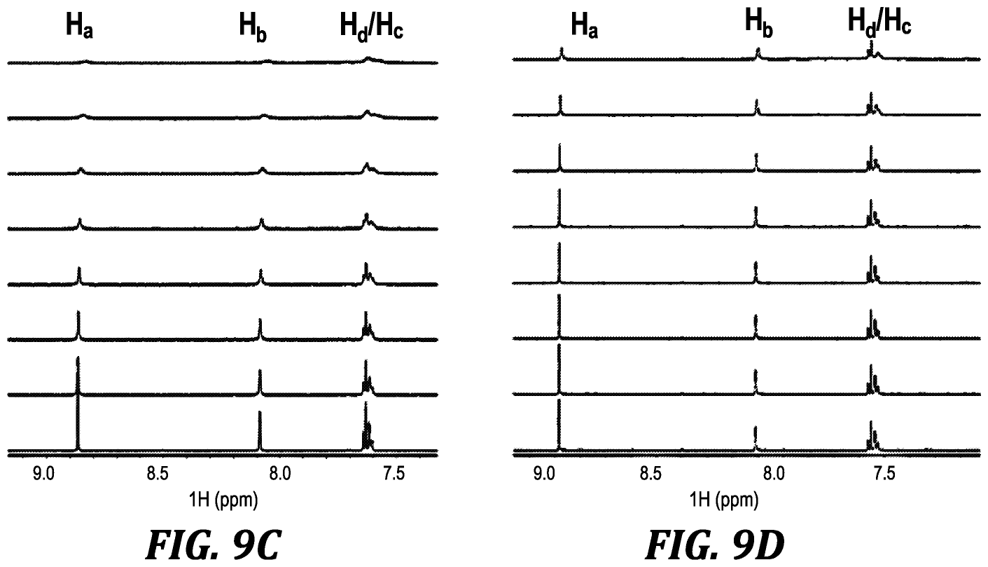

For this step, 100 μM samples of Palbociclib were prepared in high salt screening buffer and aliquoted. Each sample was titrated with either HIV TAR RNA or the pre-miR-21 hairpin over the same RNA concentration range (0.1-5 μM). NMR data were collected and processed as described in methods with all spectra normalized to the non-binding internal reference (DSA). For both HIV TAR (FIG. 9C) and pre-miR21 (FIG. 9D), the free reference spectrum is shown at the bottom with increasing RNA concentrations (0.1-5 μM) stacked on top. The rapid decrease in the Palbociclib NMR signal upon the addition of HIV TAR demonstrates high affinity, whereas changes in ligand signal are only observed for the highest concentrations of pre-miR-21. The binding affinity can be quantified using curve fitting methods (FIG. 9B) by plotting the signal intensity as a function of RNA concentration. However, the method is better suited as a qualitative ranking tool to select preferred compounds to move forward in a screening cascade, because the molecular weight, shape and size of the target affect the results to some extent, in addition to affinity.

Because the low salt buffer conditions used in the initial screen maximizes the number of hits, the follow up screening conducted in high salt screening buffer is necessary to determine if even relatively weak compounds possess RNA binding specificity and have the binding properties required to retain an interaction under conditions comparable to the cell, and are therefore suitable for more time consuming follow-up studies.

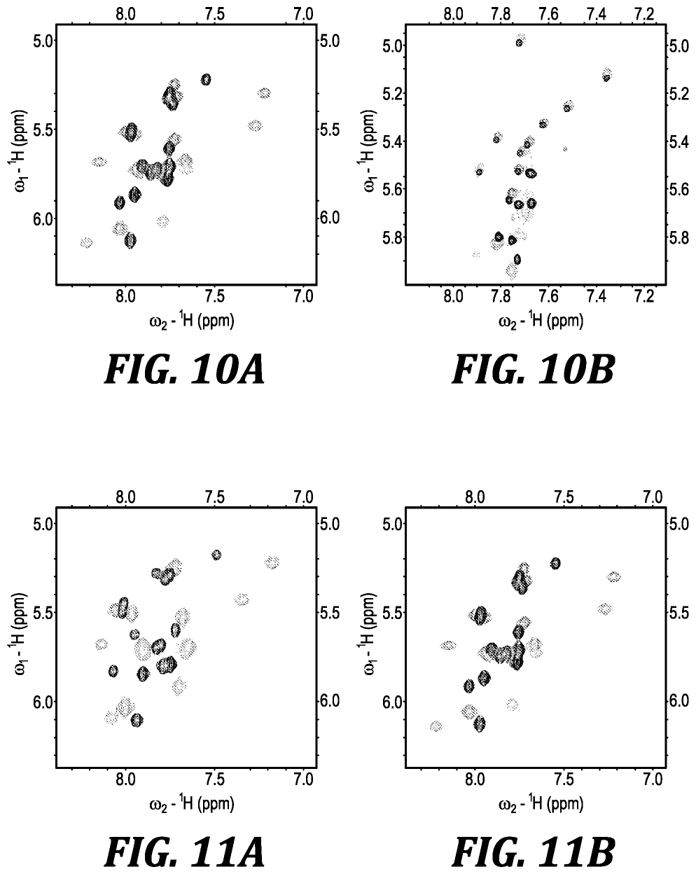

Stage 3. The third stage in the screening cascade involves monitoring changes in the NMR spectra of the RNA target to identity where on the target the compound binds and examine the structural characteristics of the interaction. This step is conducted in the same high salt buffer used for screening, prepared either in 95% H₂O/5% D₂O (water buffer) or 99.99% D₂O (D₂O buffer) to monitor different classes of proton resonances (FIGS. 10A and 10B). Uniform labeling of bases with ¹³C and ¹⁵N can also be performed, but this is relatively expensive.

For proteins, these mapping experiments are used to identify a well-defined binding site for a small molecule on the protein surface. However, for RNA, which is typically more dynamic than proteins, target-based approaches can pick up large changes in RNA structure at sites distinct from ligand binding locations as well. Therefore, in stage 5 of this approach a structure-based method, such as ¹H-¹H NOESY which measures specific interactions between the small molecule and RNA, was incorporated to address this important point. Identifying a ligand with high enough affinity that a NOESY spectrum would yield intermolecular NOEs is challenging, and relatively large sample requirements are needed as well. For these reasons, stages 1-4 were used to prioritize the best candidates for more thorough structural investigation (stage 5).

An example of the target-based detection methods is demonstrated in FIGS. 10A and 10B, where a D₂O buffer TOCSY experiment is shown for both HIV TAR (A) and pre-miR-21 (B), and the spectra of the same RNAs once fully titrated with small molecule Palbociclib. The large chemical shift changes for HIV TAR and sharp regular spectral features demonstrate the compound binds strongly (well below uM) and induces large structural changes in the RNA. These large changes are not observed for pre-miR-21, where only small chemical shift changes are observed instead, and the line shape is irregular, varying between different resonances because of differences between free and bound chemical shifts, relative compound residence time, for different nucleotides, consistent with much weaker binding. Clearly, Palbociclib binds much more strongly to HIV TAR than to pre-miR-21 as indicated by the larger chemical shift changes. The binding of Palbociclib to HIV TAR is robust to relatively large changes in pH (FIGS. 11A and 11B).

The large changes in the NMR spectra observed for HIV TAR bound to Palbociclib are indicative of a high affinity complex, with K_{D} in the nM range (we later show the affinity to be about 100 nM). The discovery of ligands with such high affinity for RNA is rare and generally requires large synthetic programs or screening campaigns. Many compounds with reported high affinity, as measured by other methods, fail to produce such large chemical shift changes in the target, when examined by NMR, probably because the interactions are driven by non-specific electrostatic contacts.

Stage 4. The fourth stage in the screening cascade quantifies binding using an orthogonal biophysical method. The small size of RNA makes it less suitable for techniques that rely on attachment to solid supports, leading to artifacts due both to the small size of the molecule and the interaction between the RNA, the small molecules and the support. Thermodynamic approaches using melting or denaturing approaches can overcome these issues but are time and material consuming.

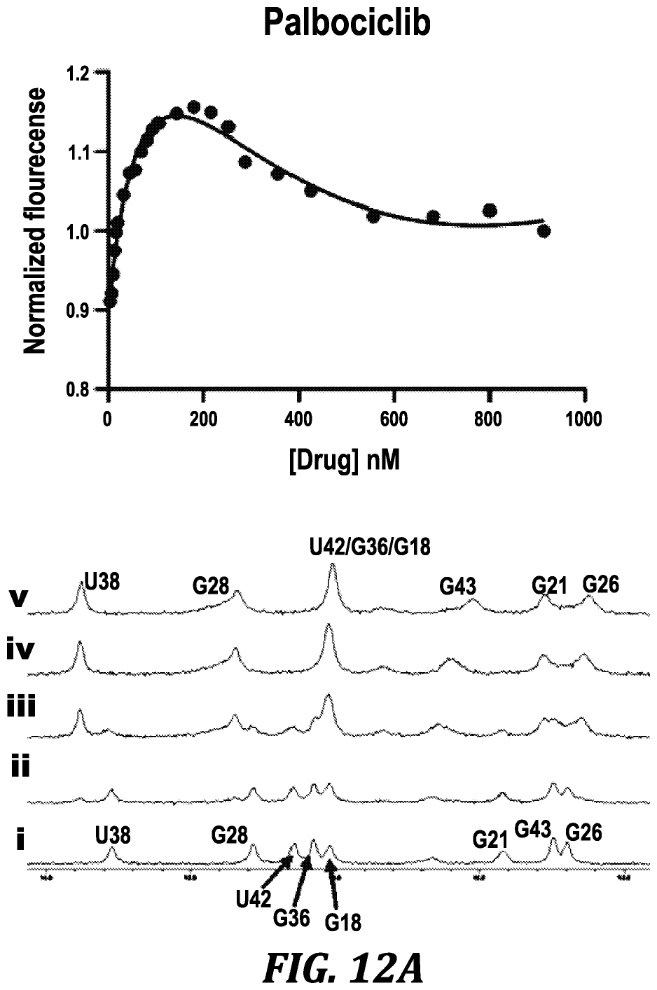

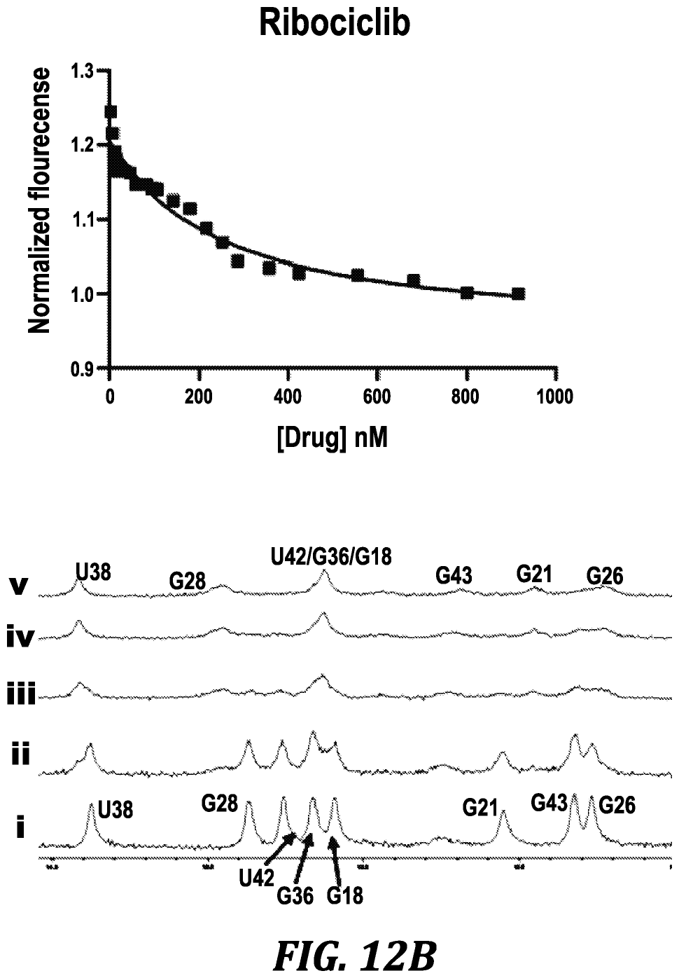

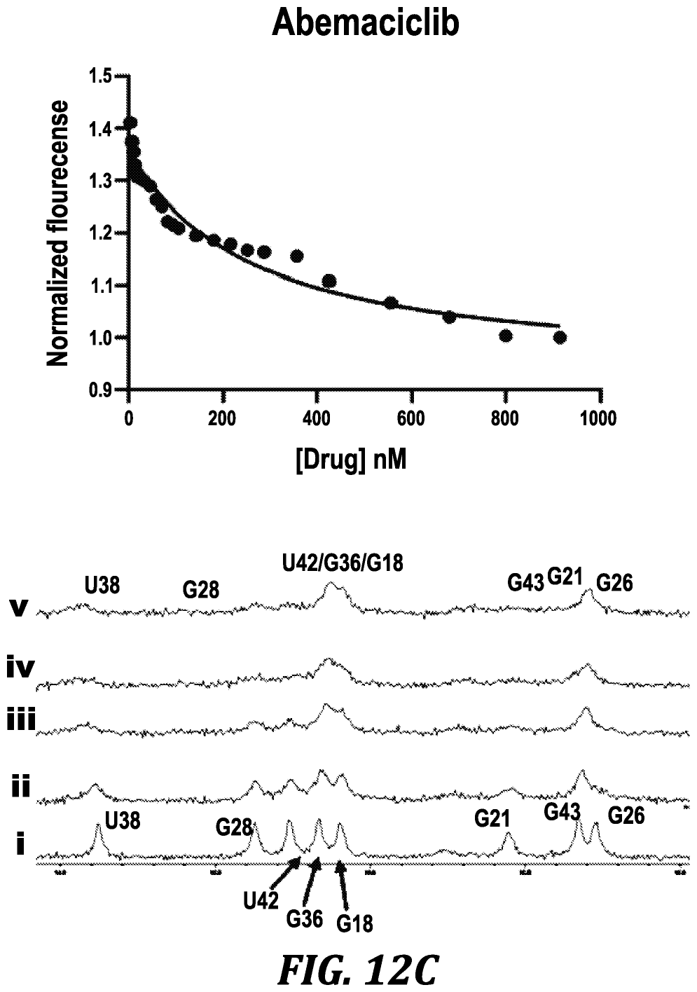

Fluorescent-based approaches provide reliable measurements and high throughput. To quantify binding of the compounds to HIV TAR, a 2-amino purine method was used. In this assay, U25 on HIV TAR was substituted with 2-aminopurine and binding assays were conducted on a Horiba Tau fluorometer. The resulting binding curve of Palbociclib for HIV TAR showed a rapid increase in fluorescence suggestive of an increase in base stacking, followed by a decrease in intensity when ligand concentrations exceeds 200 nM, suggesting a biphasic or two-site binding mode at the higher ligand concentrations (FIG. 12A top). This behavior has been reported before for other TAR RNA ligands, including the HIV TAT peptide. The high affinity binding for Palbociclib to HIV TAR was measured to be K_{D}=104±80.2 nM, in the presence of a large excess of competitor tRNA in the solution, which was routinely used to reduce non-specific binding, when the curves were fit to a two site binding mode to account for the apparent secondary low affinity binding site. The secondary low affinity binding site was measured to have an affinity greater than 700 nM based on the curve fitting analysis.

Ribociclib and Abemaciclib, which bind to the same kinases as Palbociclib, did not show the same response as Palbociclib in the binding assays; rather, they showed a decrease in fluorescence signal suggesting the base remains unstacked during binding, similar to what was observed for neomycin binding to HIV TAR and for the weaker binding phase of Palbociclib. The affinities for Ribociclib and Abemaciclib were measured to be 215.4±56 nM and 229.8±63 nM by fitting Equation 1 (see METHODS below). The larger than expected error in all three measurements could result from non-uniform mixing or slight differences in tRNA concentrations between measurements.

The 2AP binding data mirror the NMR titration data closely both with regards to the 2D 1H-1H TOCSY spectra (FIGS. 10A and 10B and 11A and 11B) and changes in the 1D 1H imino spectra (FIG. 12A bottom). The binding constant measured from fluorescence studies is comparable, within a factor of 2, to that obtained by NMR titration and is in line with the slow chemical exchange observed in ¹H-¹H TOCSY spectra when titrating Palbociclib into HIV TAR RNA (FIG. 12A). This suggests that Palbociclib has high affinity and specificity for the HIV TAR hairpin; in fact, the affinity is comparable to what is observed for its cellular target, cdk6, for which this ligand was optimized over a decade-long effort.

Stage 5. Compounds that have made it through interrogation stages 1-4 are subjected to the final step, where a ¹H-¹H NOESY spectrum of the target RNA molecule is collected then the identified small molecule compound is titrated in. Given the amount of time it takes to prepare significant amounts of RNA samples for structure determination (2-3 days/sample or more), this stage is reserved for only the best compounds with high likelihood of observing intermolecular NOEs. The RNA is prepared typically at 0.5-1.0 mM in concentration and the small molecule concentration is at similar concentration or slightly in excess. The NOESY spectra can be collected at mixing times varying between 100 and 300 ms; intermolecular NOEs observed at shorter mixing times indicate strong and specific binding.

In summary, the invention provides a generalized approach to discovering small molecules that bind to structured RNAs. When coupled with commercially available automatic sample changers and incorporating small molecule screening mixtures, rather than single molecule screening, the method is efficient at both identifying binding compounds but also in ranking binding compounds amenable for structural analysis. As described herein, the method is demonstrated to identify new small molecules targeting TAR with low nM affinity. The approach which described herein can be applied with any commercially available or proprietary compound library and can be applied to screen any structured cellular RNA of suitable size.

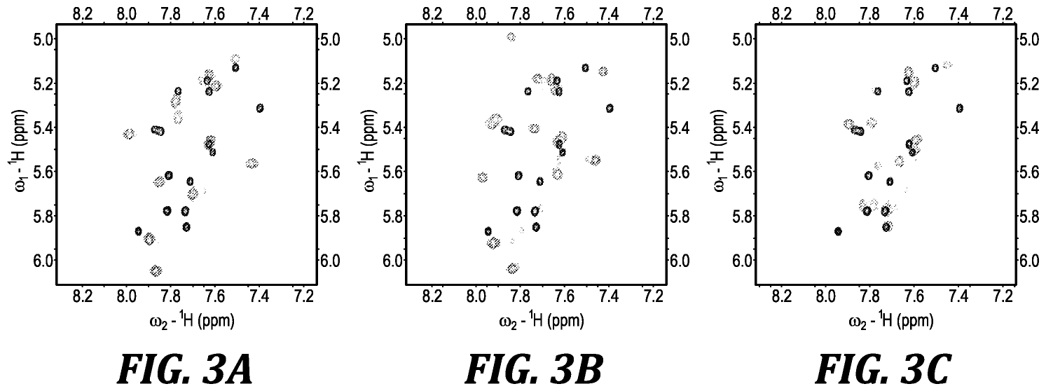

Dissection of binding requirements of cdk6 inhibitors to HIV TAR. Small molecules with high affinity for HIV TAR have been reported in the literature, but very few have the characteristics observed for Palbociclib, namely low nM binding activity and drug-like properties. The compound with the most potent binding activity and most favorable pharmacological characteristics were discovered in a systematic structure-based drug design campaign that required the synthesis of >500 compounds. Remarkably, even these highly elaborated compounds do not display the very large chemical shift changes we observe for Palbociclib (FIG. 3C vs FIG. 10A). The Palbociclib requirements for RNA binding were dissected to understand what drives its high affinity and specificity towards HIV TAR both by identifying features that promote binding to RNA, and also to investigate whether kinase binding could be separated from RNA binding.

Ribociclib and Palbociclib have very similar structure (FIG. 6), yet bind with very different affinities and physical characteristics, as revealed both by 2-AP measurements and analysis of changes in the 1D ¹H NMR imino signals (FIG. 12A). Palbociclib show two-stage binding, with a high affinity site (K_{D}=104±80.2 nM) and a second low affinity site (>700 nM); the NMR data reveal clear slow chemical shift exchange behavior, consistent with the low nM binding mode (FIGS. 10A and 11A). In contrast, Ribociclib showed a single binding event with small chemical shift changes and increased peak broadening, consistent with high nM binding and intermediate exchange behavior. These data suggest that both molecules share the same low affinity or diffuse binding site, but Palbociclib can access a second high-affinity site that is not available to Ribociclib. The similarities in the two small molecules structures (they share all the same kinase targets) do not provide immediate explanations as to what differentiates the two molecules with regards to RNA binding.

The comparison between Palbociclib and Abemaciclib is more informative; because of the larger decrease in affinity and because the 1D ¹H NMR data in FIGS. 12A and 12C suggest Abemaciclib binds non-specifically (small chemical shift changes in the imino signals, peak broadening). The decrease in affinity for the high affinity site on the RNA is likely related to the structural differences between the two small molecules. Notably, the pyrido[2,3,D]pyrimidin-7-one core structure of Palbociclib is replaced with 5-fluoro-4-(1-isopropyl-2-methyl-1H-benzo[d]imidazol-6-yl)pyrimidin-2-amine, where the 1-isopropyl-2-methyl-1H-benzo[d]imidazole fragment presumably mimics the cyclopentyl group. Furthermore, the entire 5-(piperazin-1-yl)pyridin-2-amine sub-structure in Palbociclib is replaced with a 6-((4-ethylpiperazin-1-yl)methyl)pyridin-3-amine sub-structure. These larger differences between Abemaciclib and Palbociclib, coupled with the non-specific binding to HIV TAR for Abemaciclib, suggest the cyclopentyl groups of Palbociclib and Ribociclib are important for the affinity towards HIV TAR.

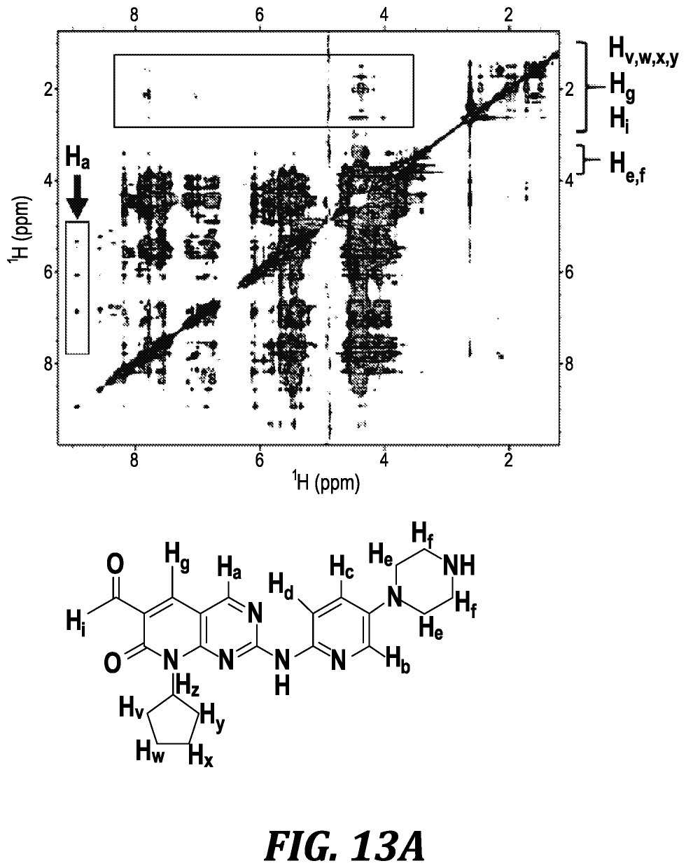

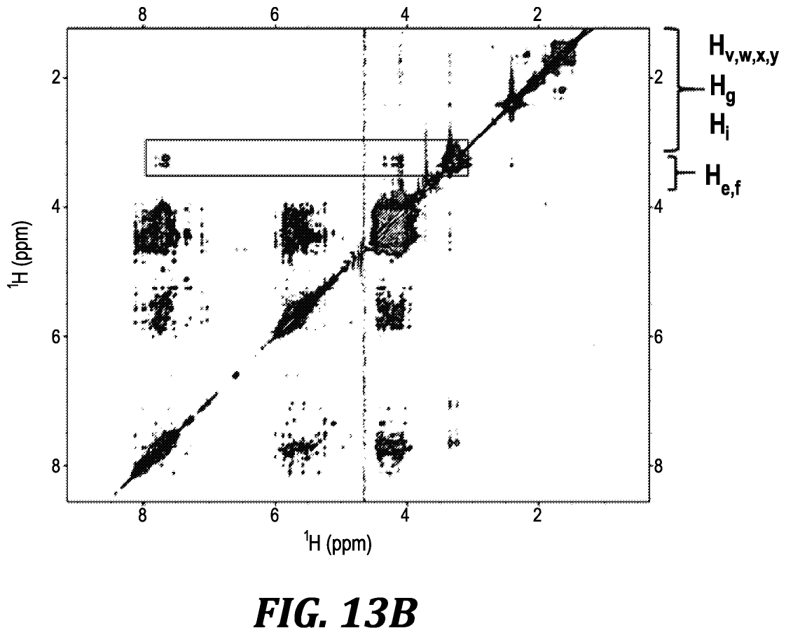

To achieve a better understanding of these differences, further studies were pursued based on these hypotheses. HIV TAR was titrated with Palbociclib and a ¹H-¹H NOESY was collected to identify direct small molecule-RNA contacts (FIG. 13A). From these data, we identified >50 intermolecular NOE interactions between Palbociclib and HIV TAR. The largest number of intermolecular NOEs involve the pyrido[2,3,D]pyrimidin-7-one core structure, including the methyl protons (H_{g},Hᵢ), the pyrimidine proton (Ha) and the cyclopentyl group on N8 (H_{v,w,x,y,z}). Intermolecular NOEs that directly report on this interaction are boxed in FIG. 13A and provide unquestionable and direct physical evidence for close contacts between this part of the small molecule and the RNA. This observation strengthens the conclusion that the interaction between Palbociclib and TAR RNA is not driven by electrostatics because it does not involve the slightly basic moiety. They also suggest that the six-membered pyrimidine ring structure in Palbociclib may facilitate closer contacts between the RNA and the cyclopentyl group, compared to the five-member pyrrole ring of Ribociclib. These data also suggest why replacing the cyclopentyl group of Palbociclib for the 1-isopropyl-2-methyl-1H-benzo[d]imidazole sub-fragment in Abemaciclib greatly reduced specific binding.

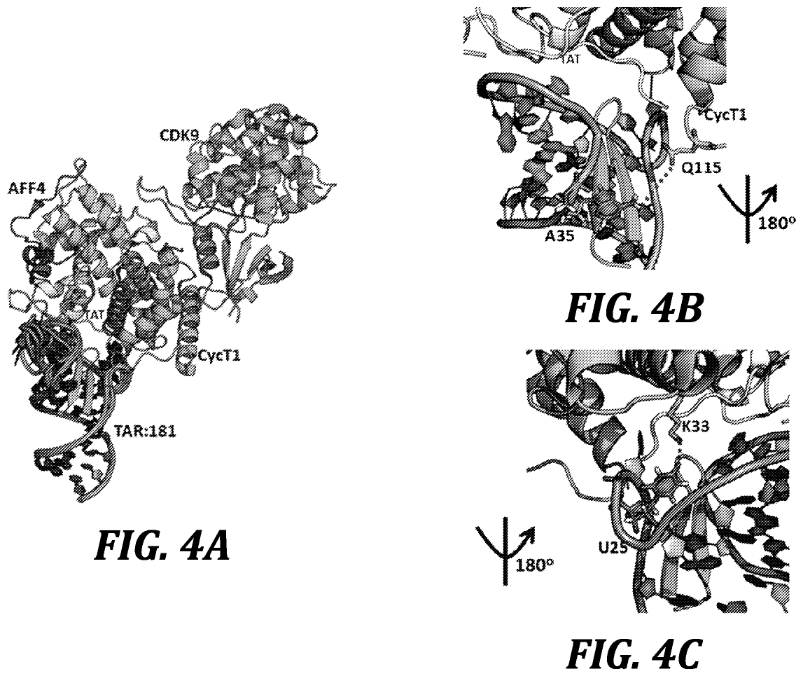

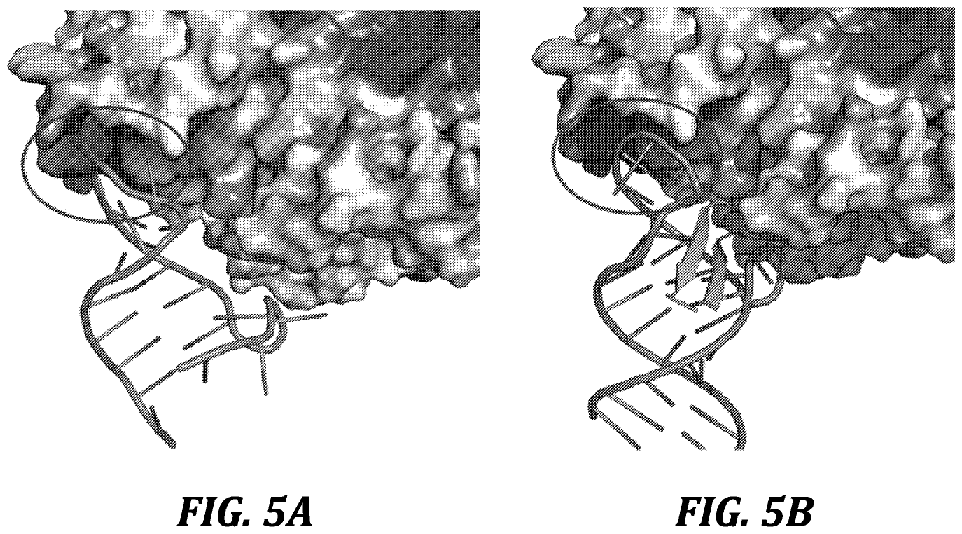

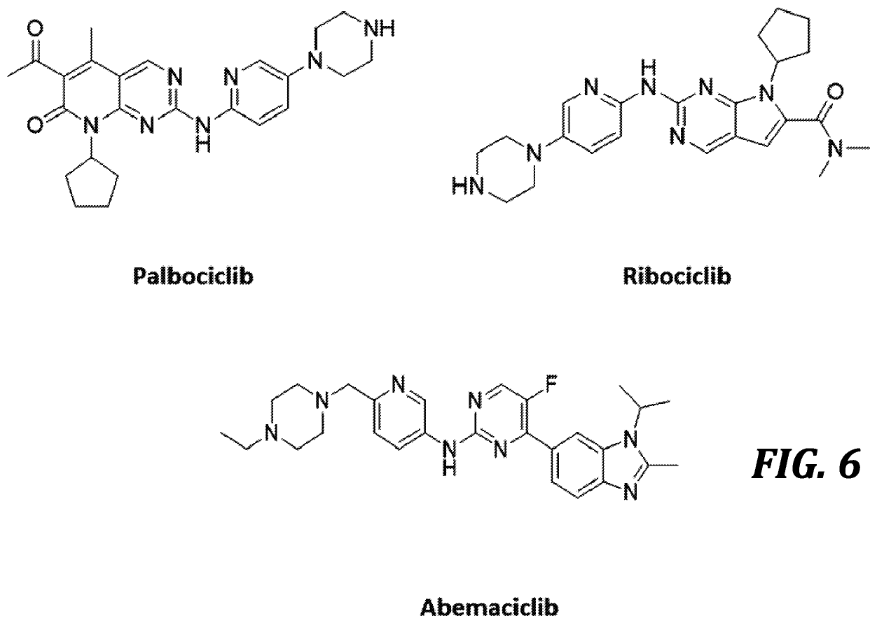

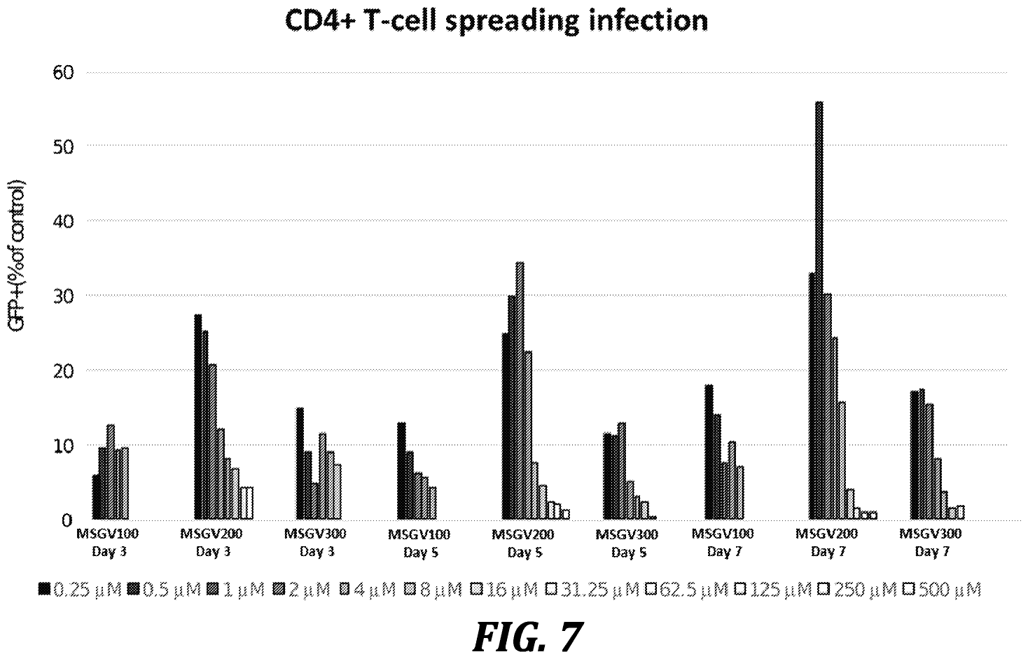

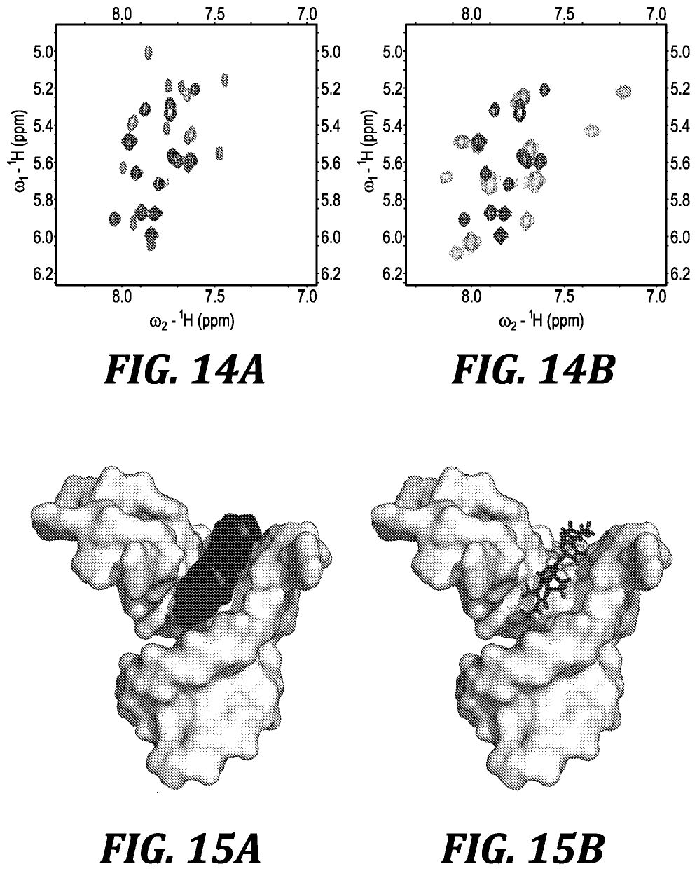

Palbociclib binds to the HIV TAR loop: the ¹H-¹H TOCSY fingerprinting method described above (FIG. 4) was used to show Palbociclib binding induces a new structure in HIV TAR, which is distinct from binding of peptides and other bulge-binding small molecules. In FIGS. 14A and 14B, the free HIV TAR, JB181 and Palbociclib TOCSY spectra are shown. The chemical shift changes induced by Palbociclib are unique and in a different direction compared to the JB181 complex, supporting the conclusion of a new structure for HIV TAR. Furthermore, the chemical shift changes between free and Palbociclib-bound TAR coupled with the intermolecular NOE pattern suggest the small molecule binds to the apical loop of HIV TAR rather than the UCU bulge, and therefore suggest that Palbociclib could inhibit the formation of the P-TEFb/AFF4/Tat/TAR complex.

To confirm the results of the fingerprint analysis (FIGS. 14A and 14B), the structure based on the intermolecular NOEs identified in FIGS. 13A and 13B was generated. This initial model of the TAR-Palbociclib complex is shown in FIGS. 15A and 15B.

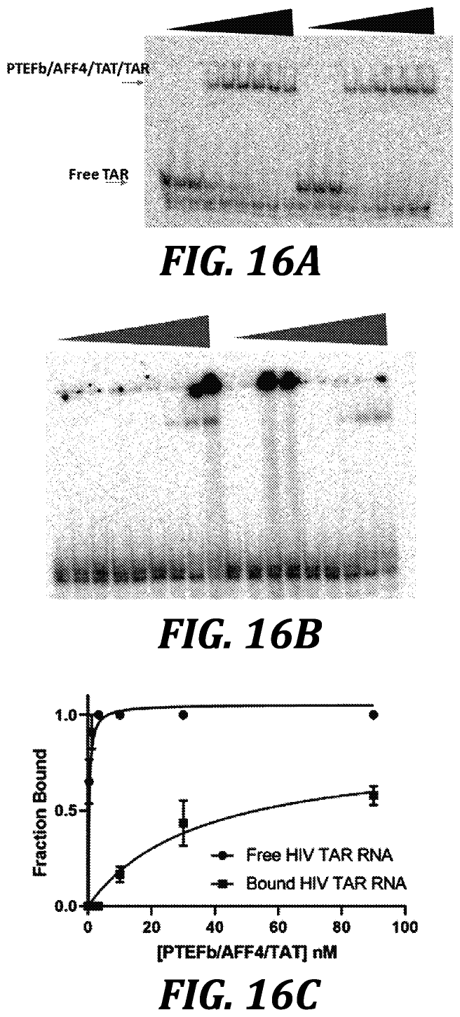

Because the structural analysis demonstrates that Palbociclib binds to the apical loop, we tested whether the compound would reduce affinity of the P-TEFb/AFF4/Tat complex for HIV TAR. The same binding assays were run as described above and compared the binding affinity of P-TEFb/AFF4/Tat to free-TAR with the binding affinity of P-TEBF/AFF4/Tat to the preformed Palbociclib-bound TAR complex (FIGS. 16A and 16B). In this assay, a 100-fold decrease in binding affinity between P-TEFb/AFF4/Tat and the preformed Palbociclib-TAR complex (K_{D} 35.6 nM) was found, compared to free TAR RNA (K_{D} 0.34 nM). The decrease in affinity toward the TAR-Palbociclib complex were comparable to mutational data, where the UGGG loop residues of TAR (K_{D} 0.23 nM) were mutated to CAAA (K_{D}>10 nM).

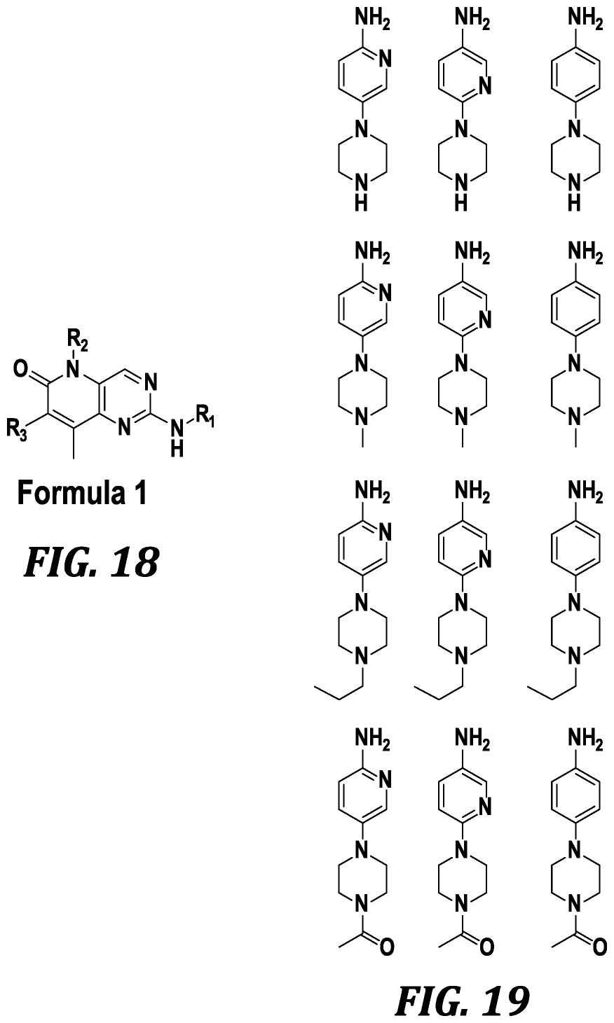

Formula 1 Compounds. The core structure of Formula 1 was derived through modeling work using Biosolveit SeeSAR package (v.7) using the publicly available Cdk6 enzyme bound to the three commercially available kinase inhibitors (Palbociclib, Abemaciclib, and Ribociclib). The Biosolveit SeeSar package was used to estimate expected decreases in apparent affinity for CDK6, but not to interrogate affinity for the RNA, since the software is not suitable for work with RNA. For Formula 1, the pyrido[2,3,D]pyrimidin-7-one core fragment of Palbociclib was reversed to generate a pyrido[3,2,D]pyrimidine-6-one core (FIG. 18) coupled to different basic R¹ substitutions (FIG. 19). Structures of these compounds are shown in Table 4.

TABLE 4
Compounds Derived from Formula 1.

| Compound | Identifier | Structure |
|---|---|---|
| 1 | MSGV-0001 | 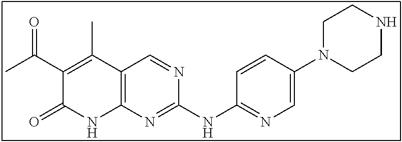 [SMILES: CC(=O)c1c(C)c2cnc(Nc3ccc(N4CCNCC4)cn3)nc2[nH]c1=O] |
| 2 | MSGV-0002 | 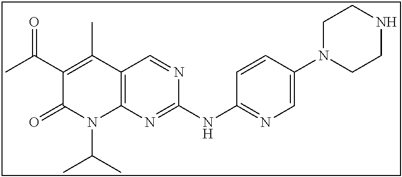 [SMILES: CC(=O)c1c(C)c2cnc(Nc3ccc(N4CCNCC4)cn3)nc2n(C(C)C)c1=O] |
| 3 | MSGV-0003 | 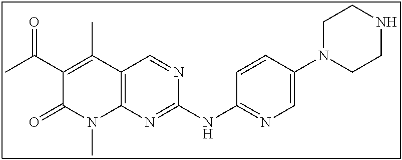 [SMILES: CC(=O)c1c(C)c2cnc(Nc3ccc(N4CCNCC4)cn3)nc2n(C)c1=O] |
| 4 | MSGV-0055 | 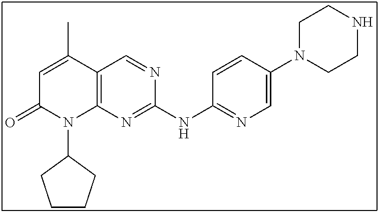 |
| 5 | MSGV-0054 | 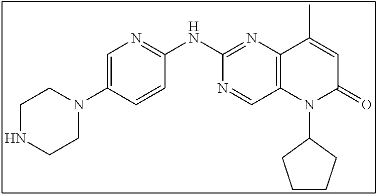 [SMILES: Cc1cc(=O)n(C2CCCC2)c2cnc(Nc3ccc(N4CCNCC4)cn3)nc12] |
| 6 | MSGV-0056 | 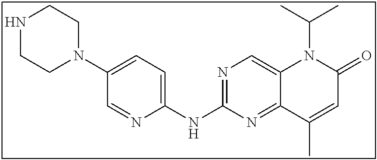 [SMILES: Cc1cc(=O)n(C(C)C)c2cnc(Nc3ccc(N4CCNCC4)cn3)nc12] |
| 7 | MSGV-0057 | 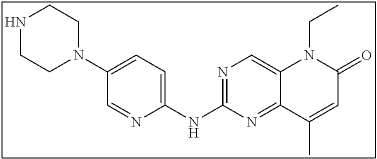 [SMILES: CCn1c(=O)cc(C)c2nc(Nc3ccc(N4CCNCC4)cn3)ncc21] |
| 8 | MSGV-0058 | 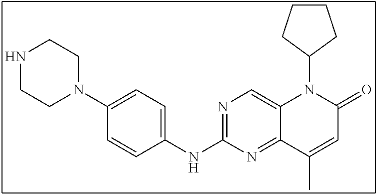 [SMILES: Cc1cc(=O)n(C2CCCC2)c2cnc(Nc3ccc(N4CCNCC4)cc3)nc12] |
| 9 | MSGV-0059 | 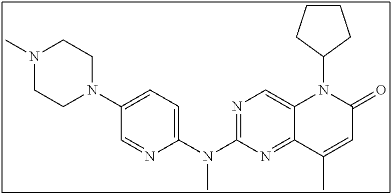 [SMILES: Cc1cc(=O)n(C2CCCC2)c2cnc(N(C)c3ccc(N4CCN(C)CC4)cn3)nc12] |

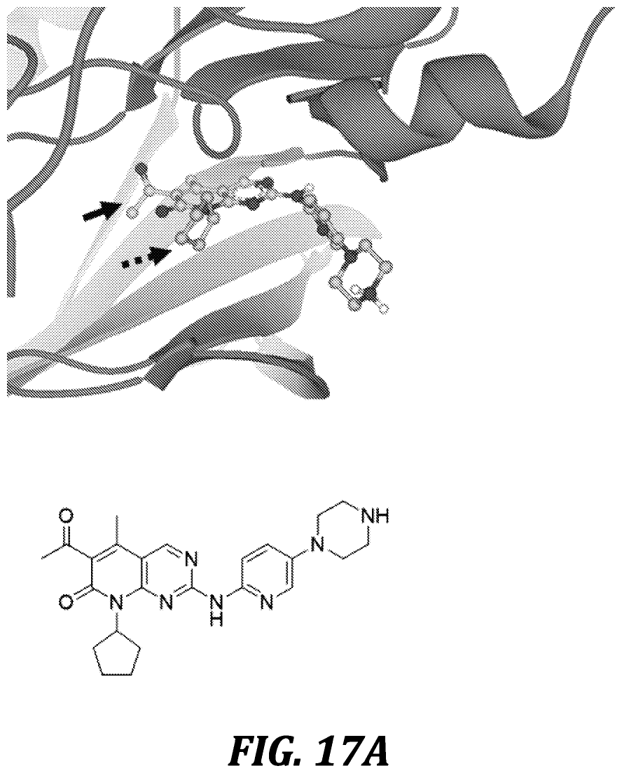

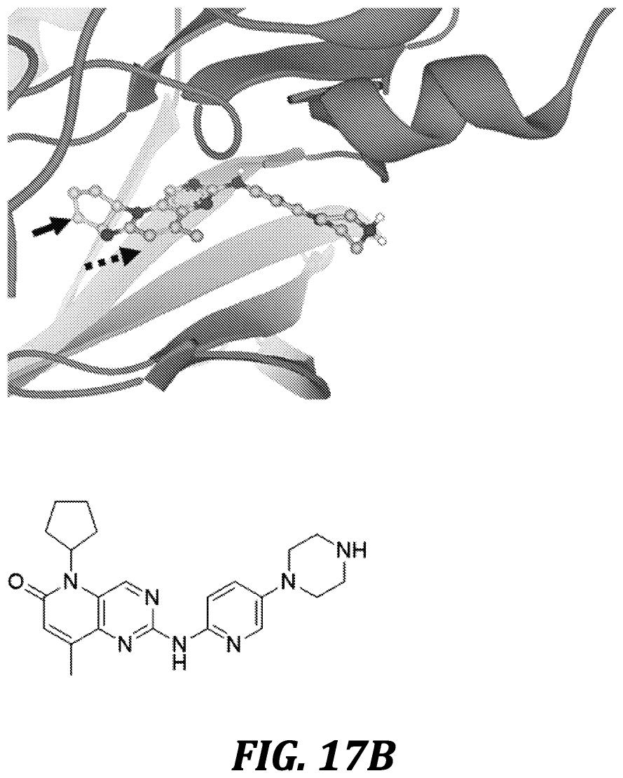

Based on modeling studies with the Biosolvit SeeSAR tools, this change in core structure would likely lead to steric clashes within the kinase active site, with the 5-(piperazin-1-yl)pyridin-2-amine generating the largest clashes (FIGS. 17A and 17B). By engineering clashes or non-optimal contacts with the CDK4/CDK6 proteins, compounds of class 1 would have greatly reduced affinity for kinases, as shown in FIGS. 17A and 17B.

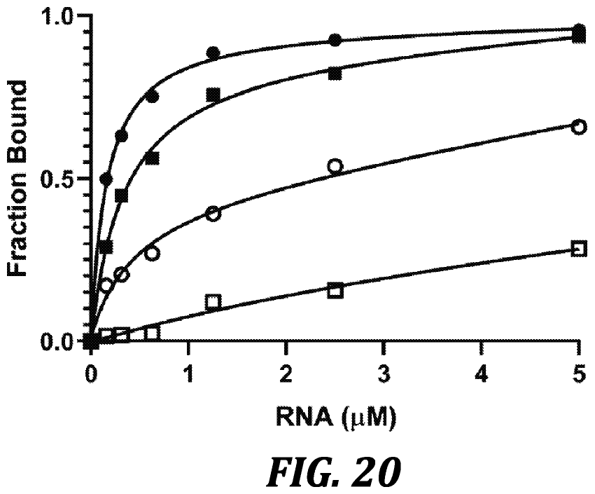

Compounds of Formula 1 display increased affinity for HIV TAR in high salt buffer, according to the ‘stage 2’ NMR screening analysis (FIG. 20). When titrating HIV TAR into a standard 100 μM sample of Compound 4 in high salt buffer, for example, we observed an even more rapid decrease in free ligand linewidth compared to what we observe with Palbociclib. The decrease in free ligand linewidth was plotted against RNA concentration, and the increase in slope of the curve with fraction bound (B) approaches 1, confirming tighter binding than Palbociclib. For proteins, which have a more compact and relatively uniform shape compared to RNA, these curves can be directly fit using equation 2 to extract binding constant measurements. For short RNAs like TAR, this assumption is also approximately valid, because the shape is not far from spherical, especially when hydration is considered. In the expression below, I_{B} is the intensity of the bound ligand peak height, I_{F} is the free ligand peak height, Pₜ is the total receptor concentration and Lₜ is the total ligand concentration. The constant c is the ratio of the bound peak width v_{B} and the free peak width v_{F}, as shown in Equation 3.

B=1−I_{B}/I_{F}=1−[1+(c[Pₜ]/{[Lₜ]+K_{D}})] Eq. 2

c=v_{B}/v_{F}−1 Eq. 3

For proteins, the bound peak width (v_{B}) can be approximated by the molecular weight of the protein multiplied by a shape-related constant pas follows:

v_{B}=p*MW Eq. 4

An RNA of the size of HIV TAR would have a diameter of about 25A, accounting for hydration, and a length of 30-35A. However, the quantitative analysis of Equations 2-4 might not be warranted when a ligand induces large changes in target structure and shape upon binding; under these circumstances, variations in the line width constant c might not reliably allow measurement of bound ligand line width v_{B}, because it could change between free and bound ligand state. While the changes in hydrodynamic shape are not likely to be large given the small size of the RNA and its shape, not unlike that of a nearly spherical ellipsoid of rotation, they are nevertheless present. Changes in this constant would lead to uncertainties in the binding affinities and potentially unstable curve fitting using Equation 2.

Even if these conservative assumptions are made, the charts obtained by plotting changes in ligand peak height vs RNA concentration provide a semi-quantitative estimate of affinity and ranking of compounds. Thus, the total change in ligand line width against RNA concentration was fitted and fitted the data using Equation 5, where Bₘₐₓ is the maximum binding capacity and represents fully titrated or fully broadened ligand signal, Rₜ is the RNA concentration and NS is the slope of the nonlinear regression (non-specific binding is assumed to be linear with respect to RNA concentration):

B=1−I_{B}/I_{F}=(Bₘₐₓ*[Rₜ])/(K_{D}+[Rₜ])+NS*[Rₜ] Eq. 5

Using Equation 5, an approximate affinity for Palbociclib and Compound 4 binding to HIV TAR of 0.335±0.05 μM and 0.15±0.01 μM, respectively, was measured within a factor of 3 of the fluorescence estimates, which has large uncertainty because of the presence of two not fully separated binding events. In both cases, Bₘₐₓ was greater than 0.9, suggesting the presence of single site-specific binding as the linear component of Equation 5 was reduced. For pre-miR21, Palbociclib binding was measured to bind with an affinity of 7.0±50.5 μM. The very large error, coupled with small decrease in ligand signal, further supports the conclusion that Palbociclib only binds to pre-miR21 very weakly and is therefore strongly selective for HIV TAR RNA.

The remaining structures of Formula 1 are shown in Table 4 along with the binary NMR screening results, and affinity measured according to Equation 5 and 2-amino purine fluorescence measurements, when available.

These data demonstrate that compounds of Formula 1 represent a novel class of RNA binding compounds that at the same time have reduced affinity for the Cdk6 kinases from which they were derived.

The present invention provides for the discovery and use of a new class of compounds that selectively bind to structured RNAs involved in the etiology of viral, bacterial and chronic disease. These new compounds were discovered through a new approach described herein. Their activity can be further optimized by rational, structure-based and medicinal chemistry approaches. Identifying drug-like molecules that bind to functional and structured RNA sequences is a rapidly growing area of research, fueled by the dramatic increase in the number of therapeutically relevant RNAs. Many biophysical methods exist to observe and quantify the interaction between structured RNAs and small molecules, and a number of high throughput methods have been reported to assay RNA-ligand interactions, such as ASMS. While these methods are effective for certain RNAs (e.g. riboswitches, aptamers, RNA enzymes), they often rely on the displacement of known RNA binding ligands, indirect chemical probing or fluorescent tagging of biologically relevant sites on the RNA. The method of the invention provides an approach amenable for medium throughput screening applications based on nuclear magnetic resonance (NMR). The NMR screening technique balance throughput with reliability in the measurement of binding interactions. One of the great advantages of NMR screening is that detection of binding can be done from either the target or ligand perspective and does not require labeling of either the small molecule or target, or identifications of ligands to be displaced. Isotopic labeling of the RNA is possible and improves spectral resolution and analysis but is not a requirement. Because individual NMR experiments can be easily nested and run on the same sample changer, throughput can be significant, thousands of molecules per week per instrument. Finally, the method also rapidly provides structural information on the site of direct contact between target and small molecule, which can be used to rapidly derive structure-activity relationships without resorting to expansive synthetic efforts, as demonstrated here.

The method described herein has been used for a specific purpose, the targeting of an RNA structure responsible for activation of transcription of a latent integrated retrovirus. However, the method is agnostic to RNA structure or biological function. Compounds with Formula 1 have improved affinity and selectivity towards HIV TAR with decreased affinity toward the enzymes that the original hit molecules target with potent activity.

### Methods

RNA stem-loop sequences used for evaluating compound selectivity are summarized in Table 3.

TABLE 3
RNA stem-loop sequences used for evaluating
compound selectivity.

| | |
|---|---|
| miR-21 | GACUGAUGUUGACUGUUGAAUCUCAUGGCAACACCAGUC |
| | (SEQ ID NO: 4) |
| HIV | GGCAGAUCUGAGCCUGGGAGCUCUCUGCC |
| TAR: | (SEQ ID NO: 5) |
| 7SK SL4 | GGCAAAUGAGGCGCUGCAUGUGGCAGUCUGCCUCUCUUUGCC |
| | (SEQ ID NO: 6) |

RNA Transcription. All RNAs for ligand screening and structural or biochemical work were prepared in house using in vitro transcription on a large scale (typically 10 mL). RNA transcription and purification protocols used DNA oligonucleotide templates (IDT) and T7 RNA polymerase purified. Briefly 1 mL of 8 μM TOP DNA (5′-CTATAGTGAGTCGTATTA-3′) (SEQ ID NO: 7), corresponding to the phage T7 RNA polymerase promoter region, was annealed to 80 μL of 100 μM template sequences with 13 mM MgCl₂, heated to 95° C. for 4 min then allowed to anneal to room temperature over 20 min. The annealing mixture was incubated with 5 mM of each of the four NTPs (ATP, GTP, UTP and CTP, from Sigma), 1× transcription buffer, 8% PEG-8000 and 35 mM magnesium chloride with 0.4 mg/mL T7 RNA polymerase expressed and purified using established methods. For transcription of isotopically labeled RNAs used for structure determination, ¹³C/¹⁵N enriched NTPs (Isotec) or ²H enriched NTPs (Cambridge Isotope) were substituted for the unlabeled NTPs, as desired.

All RNA samples are purified from crude transcriptions by 20% denaturing polyacrylamide gel electrophoresis (PAGE), electro-eluted and concentrated by ethanol precipitation. The samples are re-dissolved in 12 mL of high salt wash (700 mM NaCl, 200 mM KCl, in 10 mM potassium phosphate at pH 6.5, with 10 μM EDTA to chelate any divalent ions), concentrated using Centriprep conical concentrators (3,000 kDa MWC, Millipore). The RNA was then slowly exchanged into low salt storage buffer (10 mM potassium phosphate at pH 6.5, with 10 mM NaCl and 10 μM EDTA). Prior to NMR experiments, all RNA samples were finally desalted using NAP-10 gravity columns, lyophilized and redissolved in ‘screening buffer’ or ‘structure buffer’ (see below), then annealed by heating for 4 min to 90° C. followed by snap cooling at −20° C. For experiments investigating non-exchangeable protons, samples were lyophilized to dryness and dissolved into 99.99% D₂O. Samples used to study exchangeable protons were dissolved instead in 95% H₂O/5% D₂O.

Small molecule hit identification and tiered approach to ligand-detected NMR screening. In order to identify small molecules that bind to RNA and quantify the strength of the interaction, a tiered approach to NMR screening involving both ligand- and target-detected NMR experiments was used. The first stage of the screening cascade involves a binary bind/no bind decision filter, using experiments conducted in low salt buffer. Compounds were first dissolved to 1-10 mM in DMSO, depending on their solubility, then prepared to 100 μM in 490 μL low salt screening buffer (50 mM bis-Tris, pH 6.5, in 50 mM d₉-deuterated bis-tris buffer at pH 6.5 containing 11.1 μM sodium 4,4-dimethyl-4-silapentane-1-sulfonate (DSA) as chemical shift reference, all dissolved in 99.99% D₂O). The non-binding internal DSA reference allows us to directly compare the two spectra (in the absence or presence of RNA) to identify binding by decreases in the NMR signals of the ligands due to increased rotational correlation time of the ligand when it binds to RNA. The 9 protons on the internal reference also provide a control for ligand concentration. All hit screening was conducted using the 1D-¹H NMR excitation sculpting water suppression scheme (zgesgp, Bruker); a free ligand reference spectrum was collected followed by titration in 10 μL steps of 500 μM RNA stock solution (10 μM final RNA concentration in the NMR tube). Each experiment was collected with 16 scans, 16 k data points with a recycle delay of 1.0 sec to minimize initial screening time and increase throughput; each NMR experiment required 1.5 min in total, for an overall acquisition time or 4 min, including experimental setup (lock, tune, shimming). Screening was conducted on 500/600/800 Mhz instrumentation with or without a 24-samples automated sample changer.

Compounds that showed binding at the first binary bind/non-bind stage of screening were further examined by generating titration curves under high salt buffer that mimic the conditions prevalent in the cell. A 10 mL stock of each compound at 100 μM concentration was dissolved in high salt screening buffer (50 mM d₉-deuterated bis-Tris buffer, at pH 6.5, containing 11.1 mM DSA, 200 mM NaCl, 50 mM KCl and 4 mM MgCl₂). Compounds were divided into 12 1.5 mL microcentrifuge tubes at 490 μL each and titrated with 10 μL of target RNA (0.5 to 1000 μM). The final RNA concentration in each tube increased from 0.01 μM (10 nM) to 20 μM, and a tube with no RNA was used as control. Binding is detected by a decrease in intensity of the peaks relative to the free ligand over the concentration range of the added RNA. Affinity measurements are fit to single site binding curve models in GraphPad8.1.

Following this two-stage approach to hit identification, hits were further characterized by using 2D ¹H-¹H NOESY of 500 μM small molecule ligand in high salt screening buffer and comparing spectra of the molecule with and without 10 μM RNA. If the small molecule binds to RNA, the sign of the NOE cross-peaks changes because of increased correlation time, providing an independent way to verify the presence of a direct interaction.

Target detect NMR assays to measure RNA-ligand interactions. Transcribed RNA sequences (at natural isotopic abundance, along with ¹⁵N/¹³C enriched or selectively labeled ²H samples) are snap-cooled in 250-500 μL NMR buffer (50 mM d₉ bis-Tris pH 6.5, 50 mM NaCl) by heating to 95° C. for 4 min then placed at −20° C. until frozen. All ligand stocks were dissolved in 10-20 mM H₂O, D₂O or DMSO depending on ligand solubility. For samples dissolved in DMSO, no more that 5% final DMSO (v/v) is added to the NMR tube to minimize potential DMSO-induced unfolding of the RNA. Ligand interactions are detected by changes in chemical shift through a series of NMR experiments, including but not limited to 1D ¹H and 2D ¹H-¹H TOCSY, ¹H-¹H NOESY, ¹H-¹H EXSY, ¹⁵N-¹H HSQC, and ¹³C-¹H HSQC experiments, which are used to map the binding site of the ligand on the RNA. All pulse programs used in these experiments are pre-loaded standard pulse sequences provided by the Bruker software package. Experiments in H₂O NMR buffer were used to detect changes in exchangeable proton signals to assess the RNA secondary structure, while experiments in the D₂O NMR buffer were used to improve spectral resolution by reducing overlap of exchangeable proton signals and to detect intermolecular NOEs between ligand and RNA.

NMR structure methods. General methods for resonance assignments of RNA-small molecule complexes have been described. Briefly, NMR data were collected on Bruker 500, 600 and 800 MHz spectrometers equipped with HCN cryogenic probes. Unlabeled and uniformity ¹³C/¹⁵N labeled RNA-small molecule titration experiments were conducted at 5° C. using the 1D ¹H excitation sculpting pulse sequence for water suppression and 2D ¹⁵N-¹H HSQC experiments, respectively. Sugar pucker restraints were obtained from H1′-H2′ and H1′-H3′ peak intensities in 2D ¹H-¹H total correlation spectroscopy (TOCSY) experiments with TOCSY mixing times of 40 ms and 80 ms. Distance restrains were collected from 2D ¹H-¹H nuclear Overhauser effect spectroscopy (NOESY) spectra, collected in D₂O and H₂O NMR screening buffers at 25° C. and 4° C., respectively. NOESY spectra with mixing times of 100 ms to 350 ms were recorded to generate distance restraints for structure determination, with the intensity and volume of the H5/H6 pyrimidine cross peak recorded at 100 ms mixing time used to calibrate distances. All NMR data were processed using Bruker Topspin (3.1) or NMRPipe and visualized with Topspin, Sparky or CCPNMR.

After each RNA was fully titrated with a small molecule, initial RNA assignments for the complex were obtained by comparing TOCSY and NOESY spectra of the TAR: small molecule complex with those of TAR RNA 1. Complete assignments of the HIV TAR complex were obtained by using uniform ¹³C/¹⁵N heteronuclear labeling of the RNA and recording constant time ¹³C-edited 3D-NOESY-HSQC and ¹⁵N-edited-HSQC-NOESY experiments at 800 and 500 MHz, respectively. Small molecule resonances which overlap with RNA aromatic or sugar resonances were resolved using double filtered F1f:F2f type NOESY experiments collected in H₂O or D₂O NMR buffers. Intermolecular NOEs between the small molecule and the RNA were clearly observed and resolved in the D₂O NMR buffer.

2-AP binding assays. Fluorescence intensity binding assays were conducted using a method based on the incorporation of a single fluorescent base analogue (2-amino-purine, 2AP) in place of U25 for HIV TAR, and by monitoring changes in the reporter emission fluorescence intensity at 362 nm upon small molecule titration. Each experiment was collected in duplicate with 50 nM 2AP-TAR titrated with 5 mL of ligand stock solutions prepared by serial dilution. Methods were followed with 250×fold excess yeast tRNA added to the buffer as a competitor to reduce non-specific binding. Data were collected on a Horiba FL3-21tau Fluorescence Spectrophotometer, exported to Graphpad Prism v.8.1.1 and fit to Equation 1.

Y=Yo+((Yi−Yo)/2)*(([Rt]+[Lt]+Kd)−(sqrt((([Rt]+[Lt]+Kd)\hat{ }2)−(4*([Rt]*[Lt]))))) Eq. 1.

where Y is the measured fluorescence at 362 nm, Yi is fluorescence of the fully bound target, Yo is the fluorescence of the unbound RNA, Rt is total RNA concentration, Lt is total ligand concentration and Kd is the binding affinity.

Compound Synthesis. Commercially available small molecules, Ribociclib, Palbociclib, Amebaciclib, were purchased from Selleckchem while all custom-made small molecules were synthesized as described below.

Experimental Procedures for the Synthesis of MSGV-0054, MSGV-0056, and MSGV-0057.

The procedures for the synthesis of MSGV-0054, MSGV-0056, and MSGV-0057 are described and schematically illustrated below.

### Synthetic Scheme for MSGV-0054, MSGV-0056 & MSGV-0057

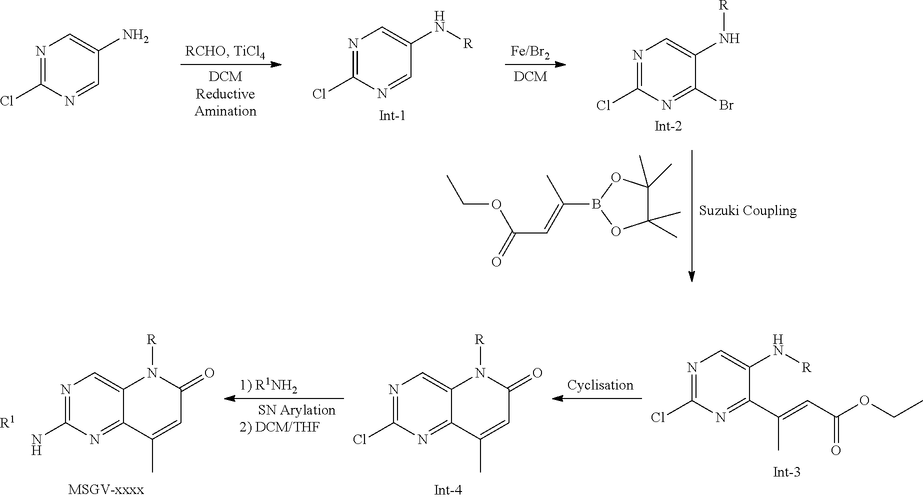

All the intermediate & final compounds were purified and fully characterized.

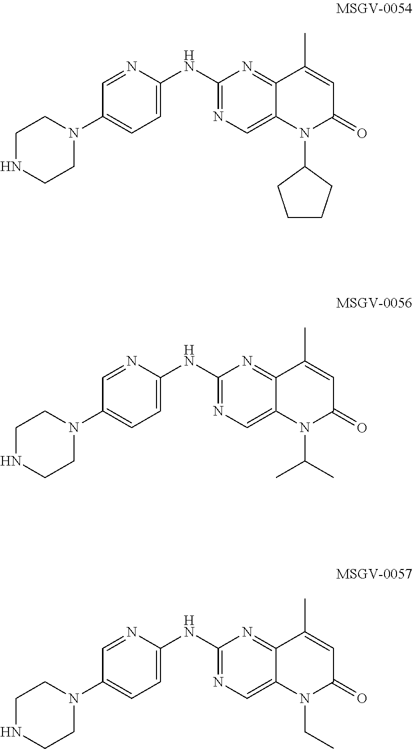 [SMILES: Cc1cc(=O)n(C2CCCC2)c2cnc(Nc3ccc(N4CCNCC4)cn3)nc12 ; Cc1cc(=O)n(C(C)C)c2cnc(Nc3ccc(N4CCNCC4)cn3)nc12 ; CCn1c(=O)cc(C)c2nc(Nc3ccc(N4CCNCC4)cn3)ncc21]

Procedure for the synthesis of Int-1: 5-Amino-2-chloropyrimidine (1 equiv.), the corresponding ketone or aldehyde (3 equiv.) and dichloromethane (0.2 M) were taken in a 100 mL RBF with a magnetic stir bar. The solution was cooled to 0° C., then a solution of TiCl₄ (1.2 equiv.) in 0.05 M of dichloromethane was added to the reaction mixture (RM) slowly over a period of 10 to 15 minutes. The RM was stirred at room temperature (RT) for 2 hours. Sodium cyanoborohydride (3 equiv.) was added in 4 equal portions over 10-minute time intervals and the RM stirred at RT for another 2 hours. The RM was cautiously quenched with water and extracted with ethyl acetate twice. The combined organic layer was dried over Na₂SO₄, filtered, and concentrated under vacuum. The crude RM was purified on silica gel using 0-30% ethyl acetate in hexanes as eluent. Relevant fractions were evaporated in vacuum to give Int-1 (Yield: 50-75%).

Procedure for the synthesis of Int-2: The corresponding Int-1 (1 equiv.), iron powder (0.1 equiv.) and dichloromethane (0.2 M) were taken in a 100 mL RBF with a magnetic stir bar. The solution was cooled to 0° C., then a solution of Br₂ (1.2 equiv.) in 0.05 M of dichloromethane was added to the reaction mixture slowly over a period of 10 to 15 minutes. The reaction mixture (RM) was stirred at room temperature (RT) for overnight (20-24 hours). The iron powder was filtered off, washed with small amount of dichloromethane and the filtrate was concentrated under vacuum. The crude RM was purified on silica gel using 0-20% ethyl acetate in hexanes as eluent. Relevant pure fractions were evaporated in vacuum to give Int-2 (Yield: 55-80%).

Procedure for the synthesis of Int-3: The corresponding Int-2 (1 equiv.), (Z)-(4-Ethoxy-4-oxo-2-buten-2-yl) boronic acid pinacol ester (1.2 equiv.), Na₂CO₃ (2.7 equiv.) and a mixture of DMF/H₂O (5:1, 0.25 M) were taken in a microwave vial with a magnetic stir bar. The solution was purged with N₂ gas for 10-15 minutes, then catalyst PdCl₂ (PPh₃)₂ (0.05 equiv.) was added to the reaction mixture (RM) and purged the N₂ gas for another 2 to 5 minutes. The RM was sealed and stirred at 90° C. for overnight (18-23 hours). Cooled the RM to room temperature (RT), filtered off and filtrate was diluted with ethyl acetate, washed with brine solution. The organic layer was dried over Na₂SO₄, filtered, and concentrated under vacuum. The crude RM was purified on silica gel using 0-30% ethyl acetate in hexanes as eluent. Relevant pure fractions were evaporated in vacuum to give Int-3 (Yield: 35-60%).

Procedure for the synthesis of Int-4: The corresponding Int-3 (1 equiv.), Cs₂CO₃ (1.2 equiv.) and DMF (0.25 M) were taken in a 100 ml RBF with a magnetic stir bar. The reaction mixture (RM) was stirred at room temperature (RT) for overnight (20-24 hours). The RM was diluted with ethyl acetate, washed with brine solution twice. The organic layer was driver over Na₂SO₄ and concentrated under vacuum. The crude RM was purified on silica gel using 0-30% ethyl acetate in hexanes as eluent. Relevant fractions were evaporated in vacuum to give Int-4 (Yield: 30-60%).

Procedure for the synthesis of final compounds MSGV-0054, MSGV-0056, and MSGV-0057: A 2-necked RBF was purged and maintained under an atmosphere of nitrogen. The corresponding aniline (2.1 equiv.) and toluene (0.20 M) were taken in RBF with a magnetic stir bar. The reaction mixture (RM) was cooled to 0° C., then LIHMDS (1 M solution in THF, 2.1 equiv.) was added to the RM over period of 2-5 minutes. After 5-10 minutes, a solution of the corresponding Int-4 (1 equiv.) in 0.05 M of toluene was added to the RM at 0° C. Then stirred the RM at room temperature (RT) for 2-4 hours. The RM was quenched with aqueous saturated NaHCO₃ solution and diluted with ethyl acetate. The organic layer was dried over Na₂SO₄ and concentrated under vacuum. The crude RM was purified on silica gel using 0-80% ethyl acetate in hexanes as eluent. Relevant pure fractions were evaporated in vacuum to give Int-5.

The corresponding Int-5 were dissolved in DCM/TFA (4:1, 0.05 M), stirred the mixture for 1-2 hours at RT. The crude reaction was concentrated and purified on HPLC using water/acetonitrile as eluent. Relevant pure peak fractions were lyophilized to give the corresponding final compounds MSGV-0054, MSGV-0056, and MSGV-0057 (Overall yield for two steps: 20-50%).

Synthetic Scheme for MSGV-0055

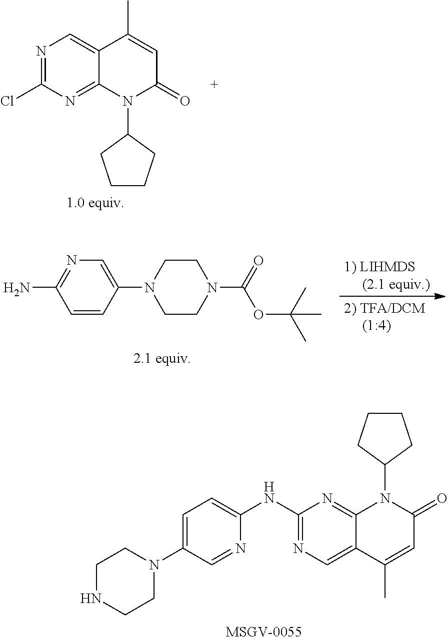 [SMILES: Cc1cc(=O)n(C2CCCC2)c2nc(Cl)ncc12 ; CC(C)(C)OC(=O)N1CCN(c2ccc(N)nc2)CC1 ; Cc1cc(=O)n(C2CCCC2)c2nc(Nc3ccc(N4CCNCC4)cn3)ncc12]

Experimental procedures for the synthesis of MSGV-0055: A 2-necked RBF was purged and maintained under an atmosphere of nitrogen. The tert-Butyl 4-(6-aminopyridin-3-yl)piperazine-1-carboxylate (2.1 equiv.) and toluene (0.20 M) were taken in RBF with a magnetic stir bar. The reaction mixture (RM) was cooled to 0° C., then LIHMDS (1 M solution in THF, 2.1 equiv.) was added to the RM over a period of 2-5 minutes. After 5-10 minutes, a solution of 2-Chloro-8-cyclopentyl-5-methylpyrido[2,3-d]pyrimidin-7(8H)-one (1 equiv.) in 0.05 M of toluene was added to the RM at 0° C. Then stirred the RM at room temperature (RT) for 2-4 hours. The RM was quenched with aqueous saturated NaHCO₃ solution and diluted with ethyl acetate. The organic layer was dried over Na₂SO₄ and concentrated under vacuum. The crude RM was purified on silica gel using 0-80% ethyl acetate in hexanes as eluent. Relevant pure fractions were evaporated in vacuum to give 4-[6-[(8-Cyclopentyl-5-methyl-7-oxo-7,8-dihydropyrido[2,3-d]pyrimidin-2-yl)amino]pyridin-3-yl]piperazine-1-carboxylic acid tert-butyl ester.

4-[6-[(8-Cyclopentyl-5-methyl-7-oxo-7,8-dihydropyrido[2,3-d]pyrimidin-2-yl)amino]pyridin-3-yl]piperazine-1-carboxylic acid tert-butyl ester was dissolved in DCM/TFA (4:1, 0.05 M), stirred the mixture for 1-2 hours at RT. The crude reaction was concentrated and purified on HPLC using water/acetonitrile as eluent. Relevant pure peak fractions were lyophilized to give the corresponding final compounds MSGV-0055 (Overall yield for two steps: 50%).

### Synthetic Scheme for MSGV-0058

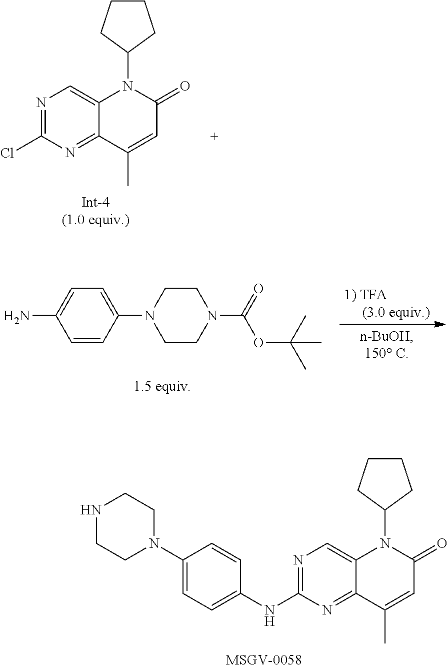 [SMILES: Cc1cc(=O)n(C2CCCC2)c2cnc(Cl)nc12 ; CC(C)(C)OC(=O)N1CCN(c2ccc(N)cc2)CC1 ; Cc1cc(=O)n(C2CCCC2)c2cnc(Nc3ccc(N4CCNCC4)cc3)nc12]

Experimental procedures for the synthesis of MSGV-0058: 1-Boc-4-(4-aminophenyl)piperazine (1.5 equiv.) and Int-4 (1.0 equiv.), TFA (3.0 equiv.) were taken in a sealed tube with n-BuOH (0.05 M). The reaction mixture (RM) was heated at 150° C. for overnight. The RM was cooled down to RM and excess TFA was quenched with triethylamine (TEA). The crude compound was purified on HPLC using water/acetonitrile as eluent. Relevant pure peak fractions were lyophilized to give the corresponding final compounds MSGV-0058 (Yield 35%).

### Synthetic Scheme for MSGV-0059

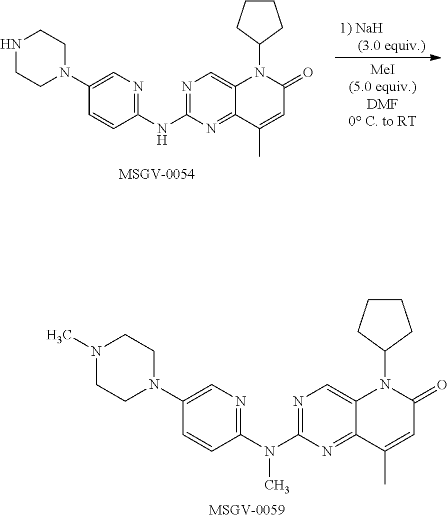 [SMILES: Cc1cc(=O)n(C2CCCC2)c2cnc(Nc3ccc(N4CCNCC4)cn3)nc12 ; Cc1cc(=O)n(C2CCCC2)c2cnc(N(C)c3ccc(N4CCN(C)CC4)cn3)nc12]

Experimental procedures for the synthesis of MSGV-0059: MSGV-0054 (1.0 equiv.), Methyl iodide (5.0 equiv.) and DMF (0.05 M) were taken in a dry 25 mL RBF with a magnetic stirrer. The reaction mixture (RM) was cooled to 0° C., sodium hydride (3.0 equiv.) was added cautiously to the RM at 0° C. The RM was slowly warmed to RT and stirred for another 2 hrs. The RM was quenched with saturated NH₄Cl solution, diluted with ethyl acetate. The combined organic layer washed with brine solution and dried over Na₂SO₄. The crude compound was purified on HPLC using water/acetonitrile as eluent. Relevant pure peak fractions were lyophilized to give the corresponding final compounds MSGV-0059 (Yield 65%).

P-TEFb-binding assay. The P-TEFb/Tat1:57/AFF4 binding assays were run as described but binding buffers contained 250× fold excess yeast tRNA. Palbociclib-bound samples were pre-prepared with 20×excess labeled HIV TAR (10 nM Palbocilib, 0.5 nM 32P TAR) and the ligand RNA complex was allowed to equilibrate for 30 min at room temp prior to titration with the pre-formed P-TEFb/Tat1:57/AFF4.complex.

Cell Assays. Spreading infection. 5×10⁶ 5.25. EGFP.Luc.M7 cells (M7-luc) grown in RPMI containing 25 mM HEPES (GE Life Sciences, Pittsburgh, Pa.), 10% FBS (Sigma, St. Louis, Mo.), and 100 μg/mL normocin (Invivogen, San Diego, Calif.) were infected with the HIV-1 strain NL43-GFP-IRES-Nef (NLgNef) that expresses GFP and Nef on a bicistronic Nef mRNA at a low multiplicity of infection (<0.01 infectious units/cell). 24 hours later, a 2-fold dilution series from 500 μM to 0.25 μM of Palbociclib, Ribociclib, and Abermaciclib were added to 5×10⁴ cells/well in a 96-well plate in triplicate. At 72, 120, 168 hours post drug addition, the percentage of cells expressing GFP was determined by flow cytometry via BD Fortessa SORP (BD, Franklin Lakes, N.J.). Flow cytometry data were analyzed using WinList 3D (Verity Software House, Topsham, Me.). Graph and statistics were generated using GraphPad Prism software (GraphPad, LaJolla, Calif.).

While illustrative embodiments have been illustrated and described, it will be appreciated that various changes can be made therein without departing from the spirit and scope of the invention.

1. A compound having formula (I): 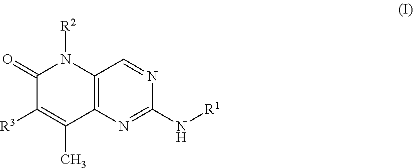
or a pharmaceutically acceptable salt thereof,
wherein R¹ is selected from the group consisting of 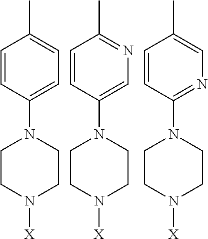
wherein X is hydrogen, C1-C6 alkyl, or C(═O)C1-C6 alkyl;
R² is selected from the group consisting of hydrogen, C1-C6 alkyl, and C3-C6 cycloalkyl; and
R³ is selected from the group consisting of hydrogen and C(═O)C1-C6 alkyl.

2. The compound of claim 1, wherein R¹ is

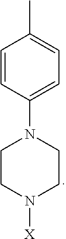

3. The compound of claim 1, wherein R¹ is

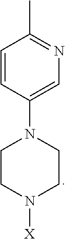

4. The compound of claim 1, wherein X is selected from the group consisting of hydrogen, methyl, ethyl, n-propyl, i-propyl, and C(═O)CH₃.

5. The compound of claim 1, wherein R² is selected from the group consisting of hydrogen, methyl, ethyl, n-propyl, i-propyl, cyclopropyl, cyclobutyl, cyclopentyl, and cyclohexyl.

6. The compound of claim 1, wherein R³ is selected from the group consisting of hydrogen and C(═O)CH₃.

7. A compound selected from the group consisting of 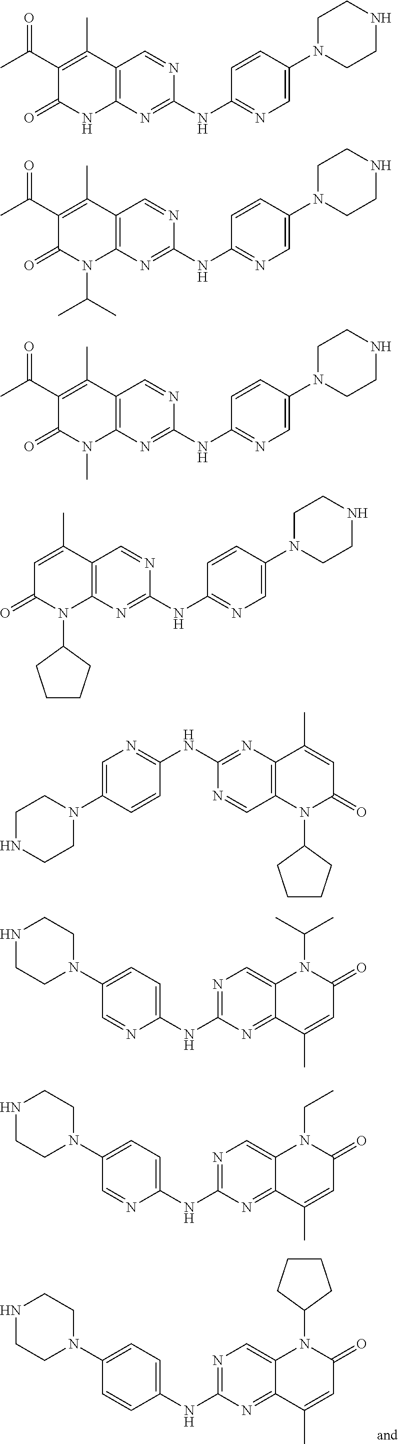 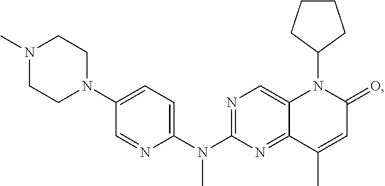 [SMILES: Cc1cc(=O)n(C2CCCC2)c2cnc(N(C)c3ccc(N4CCN(C)CC4)cn3)nc12]
or a pharmaceutically acceptable salt thereof.

8. A pharmaceutical composition, comprising a compound of claim 1, or a pharmaceutically acceptable salt thereof, and a pharmaceutically acceptable carrier.

9. A method for inhibiting the binding of human positive transcription elongation factor complex (P-TEFb) to HIV-1 trans-activation response element (HIV TAR) in a subject, comprising administering to a subject in need thereof an effective amount of a compound of claim 1.

10. A method for disrupting formation of the P-TEFb-Tat-TAR complex in a subject, comprising administering to a subject in need thereof an effective amount of a compound of claim 1.

11. A method for inhibiting miRNA processing in a subject, comprising administering to a subject in need thereof an effective amount of a compound of claim 1.

12. A method for treating a disease, disorder, or condition treatable by inhibiting miRNA processing, comprising administering to a subject in need thereof a therapeutically effective amount of a compound of claim 1.

13. A method for treating a disease, disorder, or condition treatable by inhibiting mRNA function, including but not limited to translation, alternative splicing, stability, comprising administering to a subject in need thereof a therapeutically effective amount of a compound of claim 1.

14. A method for treating a disease, disorder, or condition treatable by inhibiting the function of a noncoding RNA gene, comprising administering to a subject in need thereof a therapeutically effective amount of a compound of claim 1.

15. A method of identifying targetable and druggable RNA secondary structures in a viral RNA, viral RNA, non-coding RNA or mRNA, comprising:
contacting a primary miRNA sequence, a precursor miRNA sequence, a mRNA, viral RNA, or a noncoding RNA sequence with a ligand and determining by NMR spectroscopy whether the ligand binds to the RNA sequence.

16. A method of identifying a mRNA, miRNA or non-coding RNA ligand, comprising:
contacting an RNA sequence, comprising an RNA secondary structure, with a candidate ligand; and
determining by NMR spectroscopy whether the ligand binds to the RNA sequence, wherein binding indicates a ligand which binds to the RNA.

17. A method of identifying a miRNA ligand, comprising:
contacting a primary miRNA sequence or a precursor miRNA sequence with a candidate ligand; and
determining by NMR spectroscopy whether the ligand induces a conformation change in the primary miRNA sequence or precursor miRNA sequence, wherein the conformation change indicates the ligand binds to the primary miRNA sequence or the precursor miRNA sequence.

18. The method of claim 15, wherein the ligand is a compound having formula (I): 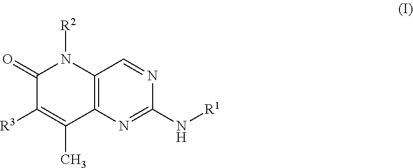
or a pharmaceutically acceptable salt thereof,
wherein R¹ is selected from the group consisting of 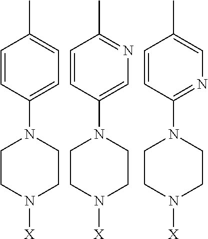
wherein X is hydrogen, C1-C6 alkyl, or C(═O)C1-C6 alkyl;
R² is selected from the group consisting of hydrogen, C1-C6 alkyl, and C3-C6 cycloalkyl; and
R³ is selected from the group consisting of hydrogen and or C(═O)C1-C6 alkyl.

19. The method of claim 16, wherein the ligand is a compound having formula (I): 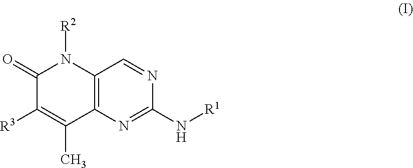
or a pharmaceutically acceptable salt thereof,
wherein R¹ is selected from the group consisting of 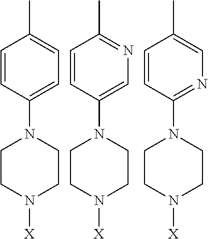
wherein X is hydrogen, C1-C6 alkyl, or C(═O)C1-C6 alkyl;
R² is selected from the group consisting of hydrogen, C1-C6 alkyl, and C3-C6 cycloalkyl; and
R³ is selected from the group consisting of hydrogen and or C(═O)C1-C6 alkyl.

20. The method of claim 17, wherein the ligand is a compound having formula (I): 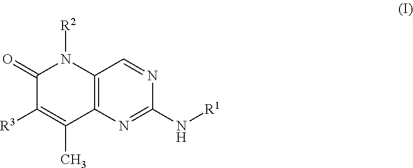
or a pharmaceutically acceptable salt thereof,
wherein R¹ is selected from the group consisting of 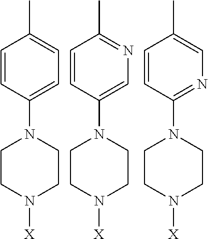
wherein X is hydrogen, C1-C6 alkyl, or C(═O)C1-C6 alkyl;
R² is selected from the group consisting of hydrogen, C1-C6 alkyl, and C3-C6 cycloalkyl; and
R³ is selected from the group consisting of hydrogen and or C(═O)C1-C6 alkyl.


SEQUENCE LISTING (7 sequences)

SEQ ID NO: 1 - protein, 14 aa, Artificial sequence
  Synthetic
  MISC_FEATURE (14)..(14): D-proline
PRVRTRGKRRIRPX

SEQ ID NO: 2 - protein, 14 aa, Artificial sequence
  Synthetic
  MISC_FEATURE (14)..(14): D-proline
PRVRTRKGRRIRIX

SEQ ID NO: 3 - protein, 14 aa, Artificial sequence
  Synthetic
  MISC_FEATURE (2)..(2): diaminobutyric acid
  MISC_FEATURE (12)..(12): norarginine (2-amino-4-guanidinobutanonic acid)
  MISC_FEATURE (14)..(14): D-proline
PXVRTRKGRRIXIX

SEQ ID NO: 4 - RNA, 39 nt, Artificial sequence
  Synthetic
gacugauguugacuguugaaucucauggcaacaccaguc

SEQ ID NO: 5 - RNA, 29 nt, Artificial sequence
  Synthetic
ggcagaucugagccugggagcucucugcc

SEQ ID NO: 6 - RNA, 42 nt, Artificial sequence
  Synthetic
ggcaaaugaggcgcugcauguggcagucugccucucuuugcc

SEQ ID NO: 7 - DNA, 18 bp, Artificial sequence
  Synthetic
ctatagtgagtcgtatta